(19) United States
(12) Patent Application Publication
(10) Pub. No.: US 2015/0135343 A1
(43) Pub. Date: May 14, 2015

(54) GTP Sensors

(71) Applicant: The Board of Regents of the University of Texas System, Austin, TX (US)

(72) Inventors: Rui Sousa, San Antonio, TX (US); Mitra Rana, San Antonio, TX (US)

(73) Assignee: The Board of Regents of the University of Texas Sy, Austin, TX (US)

(21) Appl. No.: 14/402,077

(22) PCT Filed: May 18, 2013
(86) PCT No.: PCT/US2013/041740

§ 371 Date: Nov. 18, 2014

Related U.S. Application Data
(60) Provisional application No. 61/648,998, filed on May 18, 2012.

(51) Int. Cl. G01N 33/53; C07K 14/245; C07K 14/435; G01N 33/58

(52) U.S. Cl. CPC G01N 33/5308; G01N 33/582; C07K 14/245; C07K 14/43595; C07K 2319/60; G01N 2400/00; G01N 2333/245; G01N 2333/43595
USPC 800/13; 530/350; 435/252.3; 536/23.7; 435/320.1; 435/7.8; 435/252.33; 435/252.35; 435/252.31; 435/419; 435/325

(57) ABSTRACT

Certain embodiments are directed to polypeptide sensors. Certain aspects of the invention are directed to polypeptides for sensing or detecting GTP and GTP concentrations. In further aspects, the polypeptide sensor can measure GTP concentrations, with high temporal and spatial resolution.

### STATEMENT REGARDING FEDERALLY FUNDED RESEARCH

This invention was made with government support under R21CA151128 awarded by the National Institutes of Health (NIH). The government has certain rights in the invention.

### TECHNICAL FIELD

Certain embodiments are related to the field of molecular biology.

### BACKGROUND

Biomedical researchers routinely rely on fluorescent probes to report on the concentrations of various macromolecular and small molecule constituents inside cells. These probes must have high temporal resolution (rapid changes in light emission in response to changes in the relevant ligand concentration) and spatial resolution (ability to detect differences in ligand concentration in different cellular compartments). The interest in understanding these variations in molecular concentrations is that these molecules often regulate cellular processes. The most common allosteric regulator of protein activity is guanosine triphosphate (GTP), which, through its action on the G-proteins, regulates a wide variety of cellular processes and processes relevant to many diseases including cancer.

There is currently no method to measure GTP concentrations, with high temporal and spatial resolution, inside living cells.

### SUMMARY

Certain embodiments are directed to polypeptide sensors. Certain aspects of the invention are directed to polypeptides for sensing or detecting GTP and/or GTP concentrations. The polypeptide sensor described herein can measure GTP concentrations with high temporal and spatial resolution. Spatial resolution is limited by the resolution of the microscope or means of detection. For diffraction limited optics this means ˜250 nm (2.5×10⁻⁷ M), though there are now methods that allow resolution to extend beyond this (super-resolution microscopy) and there is no reason that the sensor could not be used with such methods (i.e., the limitations are not set by the sensor but by the hardware and software of the microscope and illumination system). In regard to temporal resolution, the sensor responds to GTP addition (changes its fluorescence) within the shortest time measured, thus the temporal resolution is about 0.001, 0.1, 0.5, 1, 2, 3, or 4 seconds, including all values and ranges there between. Moreover, the intrinsic kinetics of the FeoB protein is ideal for its use as a component of a GTP sensor. Unlike most G-proteins, FeoB exhibits rapid binding and dissociation of GTP and GDP. At 35° C., the on-rate for GTP is 8×10⁵ M⁻¹sec⁻¹ and its off-rate is 12 sec⁻¹, so the sensor is responsive to changes in GTP levels at time scales as short as a few tenths of second. Consistent with its low affinity for GDP, FeoB releases GDP even more quickly: at 35° C. the rate of GDP release is too fast to measure by stop-flow methods, but was estimated at >1000/sec. Finally, FeoB has a very slow rate of GTP hydrolysis, 0.0015/sec, or about 5000-fold slower than the rate of GTP release. FeoB represents a true rapid-equilibrium system, where the degree of saturation of its active site with GTP reflects the GTP concentration in solution and not its rate of GTP hydrolysis or GDP release (Marlovits et al., Proc Natl Acad Sci USA 2002, 99, (25), 16243-48).

A polypeptide sensor described herein works by coupling the conformational changes induced in a GTP binding domain (e.g., FeoB) to light emission changes in a fluorescent protein (e.g., fused yellow fluorescent protein (Venus)). As used herein the term “fluorescent protein” means a protein that exhibits low, medium, or intense fluorescence upon irradiation with light of the appropriate excitation wavelength. The sensors (a set of them having varying affinities for GTP) have the required signal range, dynamic range, sensitivity, and response kinetics to be used as intracellular sensor of GTP levels. The GTP binding domains have two characteristics (1) GTP binding is accompanied by significant conformational changes (which can be transmitted to the fluorescent protein), and (2) The binding kinetics are relatively fast allowing rapid exchange of the GTP upon changes in the milieu. GTP-binding domains include, but are not limited to FeoB and other bacterial protein/GTPase domains Era, CgtA, or Obg. The fluorescent protein component is sensitive to conformational change. In one aspect, the fluorescent protein is a conformation sensitive circularly permuted fluorescence protein.

Various measures can be used, but are not required, to define the GTP sensors of the invention. For example: (a) the dynamic range can be approximately 1, 2, or 3 fold, (b) the affinity (e.g., K_{d}) for the GTP can be used to indicate the sensitivity, typically the sensors are most sensitive right around their Kd values, (c) the response kinetics can be determined, typically the kinetics are in the range of 0.01 to 2 seconds or less.

Certain aspects are directed to a polypeptide comprising an amino terminal GTP binding domain coupled to a central conformation sensitive fluorescent protein domain that is coupled to a carboxy terminal guanine-dissociation inhibitor domain, wherein fluorescence of the polypeptide changes upon binding to GTP. The polypeptide can further comprise a terminal normalizer domain. The normalizer domain can be positioned at the amino or carboxy terminus. In other aspects, the fluorescence of the fluorescent protein can change differentially between two different wavelengths, e.g., the emitted fluorescent can decrease upon GTP binding at one wavelength and can increase at another wavelength. This differential fluorescence can be used to normalize the fluorescence, e.g., using a ratio of emission at two wavelengths.

In certain aspects, the polypeptide has an amino acid sequence that is 80, 85, 90, 95, or 100% identical to SEQ ID NO:1, 2, or 14. In certain aspects the GTP sensor has an amino acid sequence 80, 85, 90, 95, or 100% identical to SEQ ID NO:14.

Certain aspects are directed to a cell expressing the GTP sensor. In certain aspects the cell is in organism, or the organism comprises such a cell.

Further aspects are directed to a nucleic acid encoding the GTP sensor. In certain aspects the nucleic acid is an expression vector comprising the nucleic acid encoding the GTP sensor. In a further aspect, a cell can comprise the nucleic acid described above. In certain aspects, the nucleic acid is incorporated into the genome of a cell or is present as an extrachromosomal nucleic acid.

Certain embodiments are directed to a kit comprising a GTP sensor or a nucleic acid encoding a GTP sensor.

Other embodiments are directed to methods of using the GTP sensors. In certain aspects the methods are used to measuring the GTP concentration in a cell and can comprise (a) providing a polypeptide sensor as described herein; and (b) measuring fluorescence from the sensor to determine the presence or quantity of GTP present in a cell.

The term “operatively linked” or “operably linked” or the like, when used to describe fusion proteins, refer to polypeptide sequences that are placed in a physical and functional relationship to each other. In certain embodiments, the functions of the polypeptide components of the fusion molecule are unchanged compared to the functional activities of the parts in isolation. For example, a fluorescent protein can be fused to a GTP binding polypeptide. In this case, the fusion molecule retains its fluorescence, and the GTP binding polypeptide retains its ability to bind GTP. In certain embodiments the activities of either the fluorescent protein or the GTP binding polypeptide can be altered yet still effective relative to their activities in isolation.

Reference to a nucleotide sequence “encoding” a polypeptide means that the sequence, upon transcription and translation of mRNA, produces the polypeptide. This includes both the coding strand, whose nucleotide sequence is identical to mRNA and whose sequence is usually provided in the sequence listing, as well as its complementary strand, which is used as the template for transcription. As any person skilled in the art recognizes, this also includes all degenerate nucleotide sequences encoding the same amino acid sequence.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel.

Moieties of the invention, such as polypeptides and peptides, may be conjugated or linked covalently or noncovalently to other moieties such as adjuvants, proteins, peptides, supports, fluorescence moieties, or labels. The term “conjugate” is broadly used to define the operative association of one moiety with another agent and is not intended to refer solely to any type of operative association, and is particularly not limited to chemical “conjugation.” Recombinant fusion proteins are particularly contemplated.

The term “providing” is used according to its ordinary meaning to indicate “to supply or furnish for use.” In some embodiments, the protein is provided directly by administering the protein, while in other embodiments, the protein is provided by administering a nucleic acid encoding the protein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Schematic of the sensor design. mAmetrine 1.1 is designated as the normalizer. L(x) denote the linkers of length x between the FeoB and cpYFP proteins. Peak excitation and emission wavelengths are shown. In practice, FY and the normalizer are excited at 490 nm and ˜360-380 nm respectively and the emission collected in two different channels at the same wavelength of 525 nm.

FIG. 2. Emission spectra of AF11A in the presence and absence of GTP. The signal at 405 nm is unchanged whether GTP is present or absent while that at 485 nm is decreased. This is as expected—the 405 nm mAmetrine 1.1 is insensitive to GTP while the 485 nm FY is the core of the GTP sensor.

FIG. 3. GTP binding by AF11A sensor. The lower line is the best-fit to the normalized data from the 485 nm excitation. The middle line represents the normalized GTP-insensitive data from the 360 nm excitation. The top line is the best-fit to the GTP-independent/GTP-dependent ratio. The ratiometric analysis does not distort the data as observed by the virtually same dissociation constants (K_{d}). Taking the GTP-independent as the numerator leads to the GTP levels being directly proportional to the signal thus making the data easier to read. This ratiometric analysis also highlights that the dynamic range for the GTP sensor is ˜2-fold.

FIG. 4. Quantitation of overlay of DIC images of HEK293 cells and pseudo-colored GTP-sensitive and GTP insensitive signal for the AF-AAA construct.

FIG. 5. Quantitation of merge of the GTP sensitive and insensitive pseudo colors from MPA untreated (left) and treated (right) cells. The GTP sensor in this case was AF-Triple.

FIG. 6. Quantitative ratiometric analyses of the GTP sensors. The top two panels show the distribution of the ratiometric values in cells with and without MPA treatment for 20 hrs transfected with the AF-AAA and the AF-Triple GTP sensor constructs. The panel on the left displays the average ratiometric values for the distributions shown above. The error bars represent standard deviations. The difference observed in AF-triple is significant for a p-value of 0.005.

FIG. 7. Illustrates the response of 5a5a-GTP sensor to GTP.

FIG. 8. Illustrates the effective dissociation constant (K_{eff}) determination.

FIG. 9. Illustrates the effects of pH on 5a5a-GTP sensor.

FIG. 10. Illustrates the effect of ATP on the 5a5a-GTP sensor. The K_{eff} increases from 35±1 μM to 38±2 μM for both 1 and 2 mM ATP. There is however a slight decrease in fold-change with increasing ATP concentration.

FIG. 11. 5a5a-GTP sensor is a monomer in solution. These are two independent samples at 50 μM each. The MW comes out to 59.3 and 60.8 kDa—very close to the calculated MW of ˜58.1 kDa. The solution condition was taken as that of PBS (determines the viscosity for Diffusion Coeff calculation and hence the Rₕ) and the model as isotropic sphere (determines the MW calculation from the Diffusion Coeff via Rₕ). The % mass for both cases is 99.7 with 0.3 something else that has Rₕ around 10. The buffer for the experiment was 50 mM TrisHCl, pH7.8, 100 mM KCl, 5 mM MgCl₂.

FIG. 12. Ph effects and extent of GTP-driven changes in ratiometric fluorescent signal.

### DESCRIPTION

A major area of biomedical research is measuring how changes in the concentrations of both macromolecular and small molecule components inside cells influence cellular physiology and disease. GTP is the most common allosteric regulator of protein function so measuring local and temporal variations in its concentration and understanding how they affect cellular processes is of pressing interest. There is currently no way to measure these changes in GTP concentration and the polypeptide sensors described herein solve this problem.

A myriad of cellular processes are regulated by GTP. For instance, cancer processes are regulated by GTP (through the action of G-proteins) and are especially important in oncogenesis and cancer cell survival and proliferation. GTP sensors described herein could also be used outside cells by investigators or drug companies carrying out high-throughput screens for GTPase inhibitors since it provides a fluorescent method to measure GTP consumption in a biochemical assay.

### I. POLYPEPTIDE SENSORS

Certain embodiments are directed to fusion proteins that are used as GTP sensors. A GTP sensor is a fusion-protein comprising a fluorescent protein domain and a target binding domain(s). In certain aspects, the fluorescent protein domain and the binding domain are operatively coupled by a linker. In certain aspects, the fluorescent protein domain is a conformation sensitive circularly permuted fluorescent protein. In certain aspects the fluorescent domain is a Yellow fluorescent protein (YFP). In a further aspect, the Yellow fluorescent protein is a circularly permuted Yellow fluorescent protein (cpYFP)(SEQ ID NO:4). A circularly permuted fluorescent protein is a fluorescent protein in which the amino terminus (N) and carboxy terminus (C) portions were interchanged and reconnected by a short spacer between the original termini, see Baird et al., PNAS, 96:11241-46, 1999.

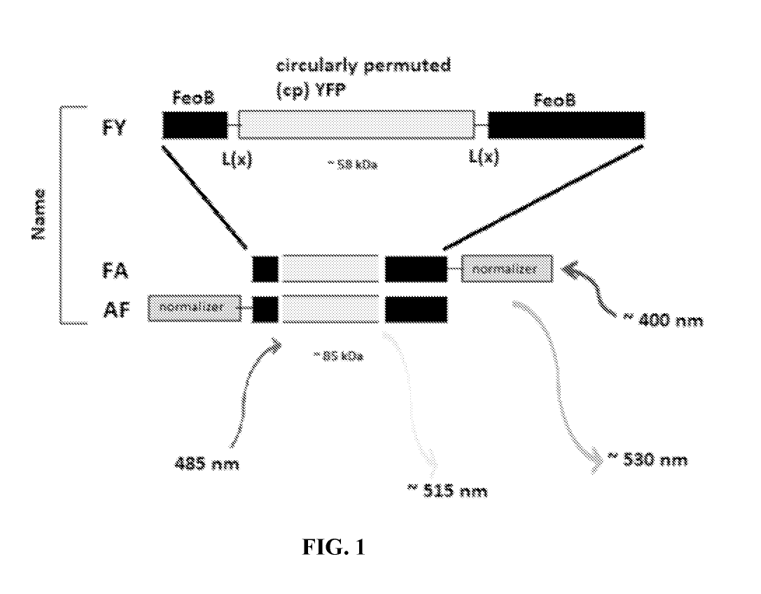

In certain aspects, the target binding domain is a GTP binding domain that undergoes a conformational change upon GTP binding. The fluorescent characteristics of the sensor are altered upon GTP binding. In certain aspects, the GTP binding domain is the G-protein domain of the FeoB protein from the bacterium Escherichia coli (SEQ ID NO:3). One example of a GTP sensor is illustrated in FIG. 1, the YFP/FeoB fusion protein is labeled FY. In one aspect an optional normalizer fluorescent protein, i.e., an FP that is not affected by a conformation change upon binding of a target, can be used for intracellular correction for expression levels and robust data analysis by ratiometric methods. A GTP-insensitive fluorescent protein can be used as a normalizer. Essentially any fluorescent protein that does not interfere with excitation of the conformation sensitive fluorescent protein can be used as a normalizer FP. Normalizers that can be used include mPlum, mCherry, and the like. In certain aspects a variant of the common GFP, mAmetrine 1.1 (SEQ ID NO:13) can be operatively coupled to either the N- or the C-terminus of the primary sensor, FY. If a normalizer is present, the sensor is a three part sensor (GTP binding polypeptide-conformational sensitive FP-normalizer FP), which, in one example, is labeled AF (if mAmetrine attached to the N-terminus) or FA (if mAmetrine attached to the C-terminus) of the primary sensor FY. In certain aspects the normalizer domain, GTP binding domain, and fluorescent domain are operatively coupled to the adjacent domain via a linker.

The AF11A GTP sensor is one example. The region comprising the protein sequence PNSGKTT (SEQ ID NO:6) of SEQ ID NO:3 has been further modified by site-directed mutagenesis to generate a series of GTP sensors with differing affinities for GTP (see Table 1 below). Consensus sequence for this region is of X₁NX₃GKX₆T, wherein in certain aspects X₁, X₃, and X₆ are any amino acid. In a further embodiment, X₁ is proline or a conservative substitution thereof, X₃ is serine or a conservative substitution thereof, and X₆ is threonine or a conservative substitution thereof. In certain aspect, X₁ is G or P, X₃ is S or A, and/or X₆ is T or S.

In one example the protein properties calculated by ProtParam (available on the World Wide Web at web.expasy.org/cgi-bin/protparam/protparam) include: Number of amino acids=753; Molecular weight=83117.1; Theoretical pI=5.31; Atomic composition—Carbon (C) 3690, Hydrogen (H) 5801, Nitrogen (N) 1001, Oxygen (O) 1135, and Sulfur (S) 24; Formula: C₃₆₉₀H₅₈₀₁N₁₀₀₁O₁₁₃₅S₂₄; Total number of atoms=11651.

Extinction coefficients: Extinction coefficients are in units of M⁻¹ cm⁻¹, at 280 nm measured in water. Ext. coefficient=69720, Abs 0.1% (=1 g/l)=0.839, assuming all pairs of Cys residues form cystines; Ext. coefficient=69220, Abs 0.1% (=1 g/l)=0.833, assuming all Cys residues are reduced.

Estimated half-life: The N-terminal of the sequence considered is M (Met). The estimated half-life is: 30 hours (mammalian reticulocytes, in vitro); >20 hours (yeast, in vivo); and >10 hours (Escherichia coli, in vivo).

Instability index: The instability index (II) is computed to be 28.17. This classifies the protein as stable.

Aliphatic index: 88.43. Grand average of hydropathicity (GRAVY): −0.324.

As used herein, an amino acid sequence or a nucleotide sequence is “substantially the same as” or “substantially similar to” a reference sequence if the amino acid sequence or nucleotide sequence has at least 85% sequence identity with the reference sequence over a given comparison window. Thus, substantially similar sequences include those having, for example, at least 85% sequence identity, at least 90% sequence identity, at least 95% sequence identity or at least 99% sequence identity. The length of comparison sequences of a fluorescent protein or fusion will generally be at least 160 amino acids, preferably at least 200 amino acids. For nucleic acids, the length of comparison sequences will generally be at least 480 nucleotides, preferably at least 600 nucleotides.

Sequence identity is calculated based on a reference sequence. Algorithms for sequence analysis are known in the art, such as BLAST, described in Altschul et al., J. Mol. Biol., 215, pp. 403-10 (1990). In one aspect, comparisons of nucleic acid or amino acid sequences are performed with Blast software provided by the National Center for Biotechnology Information using a gapped alignment with default parameters, may be used to determine the level of identity and similarity between nucleic acid sequences and amino acid sequences.

Mutants or variants may retain biological properties of the initial sensor protein (SEQ ID NO:1), or may have biological properties which differ from SEQ ID NO:1, but are still useful for detecting and measuring GTP levels. The term “biological property” of the proteins of the present invention refers to, but is not limited to, spectral properties, such as absorbance maximum, emission maximum, maximum extinction coefficient, brightness (e.g., as compared to the reference protein), and the like; biochemical properties, such as in vivo and/or in vitro stability (e.g., half-life); and other such properties (as compared to the reference protein). Mutations or substitutions include single amino acid changes, deletions or insertions of one or more amino acids, N-terminal truncations or extensions, C-terminal truncations or extensions and the like. Each polypeptide component of the sensor can be varied in such a way as to produce a variant or mutant of the protein sensor defined in SEQ ID NO:1.

Mutants or variants can be generated using standard techniques of molecular biology as described in detail in the section “Nucleic acid molecules” above. Given the guidance provided in the Examples, and using standard techniques, those skilled in the art can readily generate a wide variety of additional mutants and test whether a biological (e.g., biochemical, spectral, etc.) property has been altered. For example, fluorescence intensity can be measured using a spectrophotometer at various excitation wavelengths.

Proteins of interest can be also modified using standard techniques that includes chemical modifications, posttranslational and posttranscriptional modifications and the like. For instance, a derivative of the proteins of interest can be generated by processes such as altered phosphorylation, glycosylation, acetylation, lipidation, maturation cleavage, and the like.

The proteins of the subject invention are not naturally occurring, e.g., they are recombinant or engineered proteins. The proteins of the present invention may be present in the isolated form, by which is meant that the protein is substantially free of other proteins and other naturally-occurring biological molecules, such as oligosaccharides, nucleic acids and fragments thereof, and the like, where the term “substantially free” in this instance means that less than 70%, usually less than 60% and more usually less than 50% of the composition containing the isolated protein is some other natural occurring biological molecule. In certain embodiments, the proteins are present in substantially purified form, where by “substantially purified form” means at least 95%, usually at least 97% and more usually at least 99% pure. In other aspects, the proteins described herein can be expressed in vitro or in vivo, including expression in transgenic cells or animals.

The subject proteins and polypeptides may be synthetically produced, e.g., by expressing a recombinant nucleic acid coding sequence encoding the protein of interest in a suitable host, as described above. Any convenient protein purification procedures may be employed, wherein suitable protein purification methodologies are described in Guide to Protein Purification, (Deuthser ed., Academic Press, 1990). For example, a lysate may be prepared from the original source and purified using HPLC, exclusion chromatography, gel electrophoresis, affinity chromatography, and the like.

Also provided are fusion proteins comprising a protein of the present invention, or fragments thereof, fused, for example, to a degradation sequence, a sequence of subcellular localization (e.g., nuclear localization signal, peroximal targeting signal, Golgi apparatus targeting sequence, mitochondrial targeting sequence, etc.), a signal peptide, or any protein or polypeptide of interest. Fusion proteins may comprise for example, a fluorescent protein of the subject invention polypeptide and a second polypeptide (“the fusion partner”) fused in-frame at the N-terminus and/or C-terminus of the fluorescent protein. Fusion partners include, but are not limited to, polypeptides that can bind antibodies specific to the fusion partner (e.g., epitope tags), antibodies or binding fragments thereof, polypeptides that provide a catalytic function or induce a cellular response, ligands or receptors or mimetics thereof, and the like. In such fusion proteins, the fusion partner is generally not naturally associated with the fluorescent protein portion of the fusion protein.

### II. NUCLEIC ACID MOLECULES

The present invention provides nucleic acid molecules encoding a GTP sensor as described herein. In one embodiment the GTP sensor has an amino acid sequence of SEQ ID NO:1 and mutants or variants thereof. In certain aspects, the variants encode the amino acid substitutions defined in SEQ ID NO:7-11. Nucleic acid molecules encoding shorter or longer variants of the GTP sensor or its variants are also in the scope of the invention.

A nucleic acid molecule as used herein is a DNA molecule, such as a genomic DNA molecule or a cDNA molecule, or an RNA molecule, such as an mRNA molecule. The term “cDNA” as used herein is intended to include nucleic acids that share the arrangement of sequence elements found in mature mRNA species, where sequence elements are exons and 5′ and 3′ non-coding regions.

Nucleic acid molecules encoding the GTP sensors of the invention may be synthesized from appropriate nucleotide triphosphates or isolated from recombinant biological sources. Both methods utilize protocols well known in the art. For example, the availability of amino acid sequence information provided herein enables preparation of isolated nucleic acid molecules of the invention by oligonucleotide synthesis or by recombinant techniques. In the case of amino acid sequence information, a number of nucleic acids that differ from each other due to degenerate code may be synthesized. The methods to select codon usage variants for desired hosts are well known in the art.

Synthetic oligonucleotides may be prepared by the phosphoramidite method, and the resultant constructs may be purified according to methods known in the art, such as high performance liquid chromatography (HPLC) or other methods as described in, for example, Sambrook et al., Molecular Cloning: A Laboratory Manual, 2nd Ed., (1989) Cold Spring Harbor Press, Cold Spring Harbor, N.Y., and under regulations described in, e.g., United States Dept. of HHS, National Institute of Health (NIH) Guidelines for Recombinant DNA Research. Long, double-stranded DNA molecules of the present invention may be synthesized by synthesizing several smaller segments of appropriate complementarity that comprise appropriate cohesive termini for attachment of an adjacent segment. Adjacent segments may be linked using DNA ligase or PCR-based methods.

Nucleic acid molecules encoding the polypeptide domains described herein or their equivalent may be also cloned from biological sources or known recombinant nucleic acids.

In certain embodiments, a nucleic acid molecule of the invention is a DNA (or cDNA) molecule comprising an open reading frame that encodes the encodes the GTp sensors described herein and is capable, under appropriate conditions (e.g., cell physiological conditions), of being expressed as a GTP sensor according to the invention. The invention also encompasses nucleic acids that are homologous, substantially the same as, identical to, or variants of the nucleic acids encoding proteins described herein. The subject nucleic acids are recombinant nucleic acids, i.e., they are engineered nucleic acids not present in nature.

Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

A nucleic acid encoding a GTP sensor-related polypeptide which is an amino acid sequence variant of the sequence shown in SEQ ID NO:1 or SEQ ID NO:2 is further provided by the present invention. A nucleic acid encoding such polypeptide may show greater than 60, 70, 80, 90, 95, or 99% identity with a nucleic acid encoding SEQ ID NO: 1 or SEQ ID NO:2.

A nucleic acid encoding such polypeptide or fragments thereof may be isolated by any of a number of known methods. A fragment of a cDNA of the present invention may be used as a hybridization probe against a cDNA library from a target organism using high stringency conditions. The probe may be a large fragment, or one or more short degenerate primers. Nucleic acids having sequence similarity are detected by hybridization under high stringency conditions, for example 50° C. or above (e.g., 60° C. or 65° C.), 50% formamide, 0.1×SSC (15 mM sodium chloride/1.5 mM sodium citrate), 0.1% SDS. Nucleic acids having a region of substantial identity to the provided sequences, e.g., variants, genetically-altered versions of the nucleic acid, etc., bind to the provided sequences under high stringency hybridization conditions. By using probes, particularly labeled probes of DNA sequences, one can isolate related nucleic acids.

Variant nucleic acids can be generated on a template nucleic acid selected from the described-above nucleic acids by modifying, deleting or adding one or more nucleotides in the template sequence, or a combination thereof, to generate a variant of the template nucleic acid. The modifications, additions or deletions can be introduced by any method known in the art (see for example Gustin et al., Biotechniques (1993) 14: 22; Barany, Gene (1985) 37: 111-123; and Colicelli et al., Mol. Gen. Genet. (1985) 199:537-539, Sambrook et al., Molecular Cloning: A Laboratory Manual, (1989), CSH Press, pp. 15.3-15.108) including error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-directed mutagenesis, random mutagenesis, gene reassembly, gene site saturated mutagenesis (GSSM), synthetic ligation reassembly (SLR), or a combination thereof. The modifications, additions or deletions may be also introduced by a method comprising recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof. In some embodiments, fluorescent proteins encoded by mutant or derived nucleic acids have the same fluorescent or biochemical properties as the wild type fluorescent protein. In other embodiments, mutant or derived nucleic acids encode fluorescent proteins with altered properties.

In addition, degenerate variants of the nucleic acids that encode the proteins of the present invention are also provided. Degenerate variants of nucleic acids comprise replacements of the codons of the nucleic acid with other codons encoding the same amino acids. In particular, degenerate variants of the nucleic acids are generated to increase its expression in a host cell. In this embodiment, codons of the nucleic acid that are non-preferred or less preferred in genes in the host cell are replaced with the codons over-represented in coding sequences in genes in the host cell, wherein said replaced codons encode the same amino acid. Humanized versions of the nucleic acids of the present invention are of particular interest. As used herein, the term “humanized” refers to changes made to the nucleic acid sequence to optimize the codons for expression of the protein in mammalian (human) cells (Yang et al., Nucleic Acids Research (1996) 24: 4592-4593). See also U.S. Pat. No. 5,795,737, which describes humanization of proteins, the disclosure of which is herein incorporated by reference.

Nucleic acids encoding shorter or longer variants of the GTP sensors or variants thereof are also in the scope of the invention. As used herein, these protein variants comprise amino acid sequences with modified C-, N-, or both termini. In longer variants, the C- or N-terminus of the protein may comprise additional amino acid residues. In shorter variants one or more (usually up to 8, more usually up to 7 and preferably up to 5) amino acid residues should be eliminated from the sequence or replaced by any other amino acid residues. Such modifications do not substantially alter fluorescent properties or GTP binding properties of the proteins, but can facilitate protein folding in host cells, decrease aggregation capacity or modulate other biochemical properties of the proteins, for example, half life before degradation. In some embodiments, these modifications do not modify biochemical properties of the protein. All types of modifications and mutations noted above are performed at the nucleic acid level.

Also provided are vector and other nucleic acid constructs comprising the subject nucleic acids. Suitable vectors include viral and non-viral vectors, plasmids, cosmids, phages, etc., preferably plasmids, and used for cloning, amplifying, expressing, transferring etc. of the nucleic acid sequence of the present invention in the appropriate host. The choice of appropriate vector is well within the skill of the art, and many such vectors are available commercially. To prepare the constructs, the partial or full-length nucleic acid is inserted into a vector typically by means of DNA ligase attachment to a cleaved restriction enzyme site in the vector. Alternatively, the desired nucleotide sequence can be inserted by homologous recombination in vivo, typically by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

Also provided are expression cassettes or systems used inter alia for the production of the subject GTP sensor or for replication of the subject nucleic acid molecules. The expression cassette may exist as an extrachromosomal element or may be integrated into the genome of the cell as a result of introduction of said expression cassette into the cell. For expression, the gene product encoded by the nucleic acid of the invention is expressed in any convenient expression system, including, for example, bacterial, yeast, insect, amphibian, or mammalian systems. In the expression vector, a subject nucleic acid is operatively linked to a regulatory sequence that can include promoters, enhancers, terminators, operators, repressors and inducers. Methods for preparing expression cassettes or systems capable of expressing the desired product are known for a person skilled in the art.

Cell lines, which stably express the proteins of present invention, can be selected by the methods known in the art (e.g., co-transfection with a selectable marker such as dhfr, gpt, neomycin, hygromycin allows the identification and isolation of the transfected cells that contain the gene integrated into a genome).

The above-described expression systems may be used in prokaryotic or eukaryotic hosts. Host-cells such as E. coli, B. subtilis, S. cerevisiae, insect cells in combination with baculovirus vectors, or cells of a higher organism such as vertebrates, e.g., COS 7 cells, HEK 293, CHO, Xenopus oocytes, primary cells etc., may be used for production or expression of the protein.

When any of the above-referenced host cells, or other appropriate host cells or organisms are used to replicate and/or express the nucleic acids of the invention, the resulting replicated nucleic acid, expressed protein or polypeptide is within the scope of the invention as a product of the host cell or organism. The product may be recovered by an appropriate means known in the art.

### III. TRANSFORMANTS

The nucleic acids of the present invention can be used to generate transformants including transgenic organisms or site-specific gene modifications in cell lines. Transgenic cells of the subject invention include one or more nucleic acids according to the subject invention present as a transgene. For the purposes of the invention any suitable host cell may be used including prokaryotic (e.g., Escherichia coli, Streptomyces sp., Bacillus subtilis, Lactobacillus acidophilus, etc) or eukaryotic host-cells. Transgenic organisms of the subject invention can be prokaryotic or a eukaryotic organism including bacteria, cyanobacteria, fungi, plants and animals, in which one or more of the cells of the organism contains a GTP sensor introduced by way of human intervention, such as by transgenic techniques well known in the art.

The isolated nucleic acid of the present invention can be introduced into the host by methods known in the art, for example infection, transfection, transformation or transconjugation. Techniques for transferring the nucleic acid molecules (i.e., DNA) into such organisms are widely known and provided in references such as Sambrook et al. (Molecular Cloning: A Laboratory Manual, 3nd Ed., (2001) Cold Spring Harbor Press, Cold Spring Harbor, N.Y.).

In one embodiment, the transgenic organism can be a prokaryotic organism. Methods on the transformation of prokaryotic hosts are well documented in the art (for example see Sambrook et al. Molecular Cloning: A Laboratory Manual, 2nd edition (1989) Cold Spring Harbor Laboratory Press and Ausubel et al., Current Protocols in Molecular Biology (1995) John Wiley & Sons, Inc).

In another embodiment, the transgenic organism can be a fungus, for example yeast. Yeast is widely used as a vehicle for heterologous gene expression (for example see Goodey et al Yeast biotechnology, D R Berry et al, eds, (1987) Allen and Unwin, London, pp 401-429) and by King et al Molecular and Cell Biology of Yeasts, E F Walton and G T Yarronton, eds, Blackie, Glasgow (1989) pp 107-133). Several types of yeast vectors are available, including integrative vectors, which require recombination with the host genome for their maintenance, and autonomously replicating plasmid vectors.

Another host organism is an animal. Transgenic animals can be obtained by transgenic techniques well known in the art and provided in references such as Pinkert, Transgenic Animal Technology: a Laboratory Handbook, 2nd edition (2003) San Diego: Academic Press; Gersenstein and Vintersten, Manipulating the Mouse Embryo: A Laboratory Manual, 3rd ed, (2002) Nagy A. (Ed), Cold Spring Harbor Laboratory; Blau et al., Laboratory Animal Medicine, 2nd Ed., (2002) Fox J. G., Anderson L. C., Loew F. M., Quimby F. W. (Eds), American Medical Association, American Psychological Association; Gene Targeting: A Practical Approach by Alexandra L. Joyner (Ed.) Oxford University Press; 2nd edition (2000). For example, transgenic animals can be obtained through homologous recombination, wherein the endogenous locus is altered. Alternatively, a nucleic acid construct is randomly integrated into the genome. Vectors for stable integration include plasmids, retroviruses and other animal viruses, YACs, and the like.

The nucleic acid can be introduced into the cell, directly or indirectly by introduction into a precursor of the cell, by way of deliberate genetic manipulation, such as by microinjection or by infection with a recombinant virus or with a recombinant viral vector and the like. The term genetic manipulation does not include classical cross-breeding, or in vitro fertilization, but rather is directed to the introduction of a recombinant nucleic acid molecule. This nucleic acid molecule may be integrated within a chromosome, or it may be extrachromosomally replicating DNA.

DNA constructs for homologous recombination will comprise at least a portion of a nucleic acid of the present invention, wherein the gene has the desired genetic modification(s), and includes regions of homology to the target locus. DNA constructs for random integration need not include regions of homology to mediate recombination. Conveniently, markers for positive and negative selection may be included. Methods for generating cells having targeted gene modifications through homologous recombination are known in the art. For various techniques for transfecting mammalian cells, see Keown et al., Meth. Enzymol. (1990) 185:527-537.

For embryonic stem (ES) cells, an ES cell line may be employed, or embryonic cells may be obtained freshly from a host, such as a mouse, rat, guinea pig, etc. Such cells are grown on an appropriate fibroblast-feeder layer or grown in the presence of leukemia inhibiting factor (LIF). Transformed ES or embryonic cells may be used to produce transgenic animals using the appropriate technique described in the art.

The transgenic animals may be any non-human animals including non-human mammal (e.g. mouse, rat), a bird or an amphibian, etc., and used in functional studies, drug screening and the like.

Transgenic plants also may be produced. Methods of preparing transgenic plant cells and plants are described in U.S. Pat. Nos. 5,767,367; 5,750,870; 5,739,409; 5,689,049; 5,689,045; 5,674,731; 5,656,466; 5,633,155; 5,629,470; 5,595,896; 5,576,198; 5,538,879; 5,484,956; the disclosures of which are herein incorporated by reference. Methods of producing transgenic plants also are reviewed in Plant Biochemistry and Molecular Biology (eds. Lea and Leegood, John Wiley & Sons) (1993) pp. 275-295 and in Plant Biotechnology and Transgenic Plants (eds. Oksman-Caldentey and Barz), (2002) 719 p.

For example, embryogenic explants comprising somatic cells may be used for preparation of the transgenic host. Following cell or tissue harvesting, exogenous DNA of interest is introduced into the plant cells, where a variety of different techniques is available for such introduction. With isolated protoplasts, the opportunity arises for introduction via DNA-mediated gene transfer protocols, including incubation of the protoplasts with naked DNA, such as plasmids comprising the exogenous coding sequence of interest in the presence of polyvalent cations (for example, PEG or PLO); or electroporation of the protoplasts in the presence of naked DNA comprising the exogenous sequence of interest. Protoplasts that have successfully taken up the exogenous DNA are then selected, grown into a callus, and ultimately into a transgenic plant through contact with the appropriate amounts and ratios of stimulatory factors, such as auxins and cytokinins.

Other suitable methods for producing plants may be used such as “gene-gun” approach or Agrobacterium-mediated transformation available for those skilled in the art.

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Polypeptide Construction

One example of a GTP binding domain is the bacterial FeoB protein (SEQ ID NO:3). This protein undergoes a conformational change upon binding GTP (this may be considered the sensing and mechanical transduction part of the sensor). In certain aspects, a light-emitting circularly permuted yellow fluorescent protein (‘Venus’) (SEQ ID NO:4) is coupled to the FeoB protein. Twenty-four constructs were produced slightly varying fusion positions and linkers. Each of these 24 proteins was characterized for its ability to function as a GTP sensor and, of these 24 variants 3 functioned as needed.

A number of amino acid substitutions were introduced in to the active site of the FeoB component of the sensor for GTP, altering the affinity of FeoB for GTP. It was not known if the mutations would alter GTP affinity by the amounts required. It was also not known if such mutations would eliminate or compromise the GTP induced conformational transition on which the sensor depends. It has been seen in other systems that mutations that alter a protein's affinity for a ligand often also compromise the conformational changes induced by the ligand.

In certain embodiments the inventors attached a fluorescent protein (mAmetrine)(SEQ ID NO:13) with different spectral characteristics to one end of the sensor to act as a GTP independent normalization signal for the GTP varying fluorescence of the Venus fluorescent protein. Again, it was not known if this fusion would be functional because such tethered constructs are often unstable in vitro (the flexible linkers can be proteolyzed) and the choice of protein to use for normalization required research to identify one with appropriate brightness and spectral characteristics.

DNA and Protein Sequences Relevant for GTP-Sensor Construction.

DNA sequence for the FeoB protein synthesized with mammalian codon usage and needed restriction sites. From 5′ to 3′ the underlined sequences correspond to restriction sites for Nde, NgoMIV, SmaI, and XhoI. NdeI and XhoI were used to clone into NdeI/XhoI digested pET15b. NgoMIV and SmaI sites were used to allow insertion of cYFP DNA:

(SEQ ID NO: 15)
catatgaaaaaactgacaatcggactgatcggcaatcctaatagcggca
aaactacactgttcaaccagctgacagggtctcggcagcgggtcggaaa
ctgggccggcgtgaccgtcgagaggaagcccgggcagttctctaccaca
gaccaccaggtgacactggtcgatctgccagggacttactccctgacta
ccattagctcccagacatctctggacgaacagattgcctgccattatat
cctgagcggagacgctgatctgctgatcaatgtggtcgatgcatccaac
ctggagcggaatctgtacctgactctgcagctgctggaactgggcattc
cctgcatcgtggctctgaacatgctggacattgcagagaaacagaatat
cagaattgaaatcgatgctctgagtgcaaggctggggtgtcccgtgatc
cctctggtctcaacaaggggccgaggaattgaggccctgaagctggcta
tcgaccgctacaaagcaaacgagaatgtggaactggtccactatgccca
gcccctgctgaacgaagcagactctctggccaaagtgatgccaagtgat
attcccctgaaacagcggagatggctggggctgcagatgctggagggag
acatctacagccgggcctacgccggggaagcaagccagcatctggatgc
cgctctggctcgactgcggaatgagatggacgatcctgccctgcatatt
gctgacgcccgctaccagtgtattgctgctatttgcgatgtcgtgtcct
aactcgag.

FeoB protein sequence translated from above DNA:

(SEQ ID NO: 3)
MKKLTIGLIGNPNSGKTTLFNQLTGSRQRVGNWAGVTVERKPGQFSTT
DHQVTLVDLPGTYSLTTISSOTSLDEQIACHYILSGDADLLINVVDAS
NLERNLYLTLOLLELGIPCIVALNMLDIAEKONIRIEIDALSARLGCP
VIPLVSTRGRGIEALKLAIDRYKANENVELVHYAQPLLNEADSLAKVM
PSDIPLKORRWLGLOMLEGDIYSRAYAGEASQHLDAALARLRNEMDDP
ALHIADARYQCIAAICDVVS

The sequence encoding the cpYFP protein was amplified from the GW1CMV-Perceval vector (Berg et al., Nat Methods 2009, 6 (2), 161-6) using PCR primers with sequences identical/complementary to the underlined sequence:

(SEQ ID NO: 16)
tacaacagcgacaacgtctatatcaccgccgacaagcagaagaacggc
atcaaggccaacttcaagatccgccacaacatcgaggacggcggcgtg
cagctcgccgaccactaccagcagaacacccccatcggcgacggcccc
gtgctgctgcccgacaaccactacctgagcttccagtccaagctgagc
aaagaccccaacgagaagcgcgatcacatggtcctgctggagttcgtg
accgccgccgggatcactctcggcatggacgagctgtacaagggcggt
tccggaggcatggtgagcaagggcgaggagctgttcaccggggtggtg
cccatcctggtcgagctggacggcgacgtaaacggccacaagttcagc
gtgtccggcgagggcgagggcgatgccacctacggcaagctgaccctg
aagctgatctgcaccaccggcaagctgcccgtgccctggcccaccctc
gtgaccaccctgggctacggcctgcagtgcttcgcccgctaccccgac
cacatgaagcagcacgacttettcaagtccgccatgcccgaaggctac
gtccaggagcgcaccatcttcttcaaggacgacggcaactacaagacc
cgcgccgaggtgaagttcgagggcgacaccctggtgaaccgcatcgag
ctgaagggcatcgacttcaaggaggacggcaacatcctggggcacaag
ctggagtacaac.

cpYFP protein sequence translated from above DNA is:

(SEQ ID NO: 4)
YNSDNVYITADKQKNGIKANFKIRHNIEDGGVQLADHYQQNTPIGDGP
VLLPDNHYLSFQSKLSKDPNEKRDHMVLLEFVTAAGITLGMDELYKGG
SGGMVSKGEELFTGVVPILVELDGDVNGHKFSVSGEGEGDATYGKLTL
KLICTTGKLPVPWPTLVTTLGYGLQCFARYPDHMKQHDFFKSAMPEGY
VQERTIFFKDDGNYKTRAEVKFEGDTLVNRIELKGIDFKEDGNILGHK
LEYN.

Forward primers to amplify the cpYFP DNA were synthesized with a 5′-NgoMIV site. Primers also contained varying linker lengths between the segments corresponding to FeoB and cpYFP DNAs resulting in encoding of an additional Ser-Ala-Gly or Gly-Thr at, respectively, the N- and C-terminal points of insertion of cpYFP. By combining different forward and reverse primers, 4 different constructs could be generated from each set of 2 forward and reverse primers: Forward1: gactgccggcgttactgtttacaacagcgacaacgtc (SEQ ID NO:17) (NgoMIV site underlined) (Translation: TAGVTVYNSDNV (SEQ ID NO:18)); Forward2: gactgccggcgttactgtttctgctggttacaacagcgacaacgtc (SEQ ID NO:19) (Translation: TAGVTVSAGYNSDNV (SEQ ID NO:20)); Reverse1: ttctttacgttcgttgtactccagcttgtg (SEQ ID NO:21) (HKLEYNERKE (SEQ ID NO:22) (translation of reverse complement)); Reverse2: ttctttacgttcagtaccgttgtactccagcttgtg (SEQ ID NO:23) (HKLEYNGTERKE (SEQ ID NO:24) (translation of reverse complement)).

Primers were also synthesized with different lengths of flanking DNA encoding the FeoB sequence so as to result in insertion of cpYFP at different points between residues FeoB residues 36 and 42 of SEQ ID NO:3 (i.e., between residues 36/37, 37/38, 38/39, 39/40. 40/41, or 41/42). The primers specified above, for example, generate PCR products that, following cleavage by NgoMIV and ligation into NgoMIV/SmaI cut FeoB DNA result in a DNA encoding a protein in which the cpYFP is inserted between FeoB residue 38 and 39, either with or without ‘SAG’ or ‘GT’ linkers N- and C-terminal to the insertion point: FeoB-AGVTV (SEQ ID NO:91)(+/−SAG)-cpYFP-(+/−GT)ERKE(SEQ ID NO:92)-FeoB.

Using the primers below, 4 fusions were constructed like those above but with the cpYFP inserted between FeoB residues 36 and 37: Forward1: gactgccggcgtttacaacagcgacaacgtc (SEQ ID NO:25) Translation: TAGVYNSDNV (SEQ ID NO:26)); Forward2: gactgccggctctgctggtgtttacaacagcgacaacgtc (SEQ ID NO:27) (Translation: TAGVSAGYNSDNV (SEQ ID NO:28); Reverse1: ctccttcctctcgacggtgttgtactccagcttgtg (SEQ ID NO:29) (Translation: HKLEYNTVERKE (SEQ ID NO:30); Reverse2: ctccttcctctcgacggttgttccgttgtactccagcttgtg (SEQ ID NO:31) (Translation: HKLEYNGTTVERKE (SEQ ID NO:32)).

A total of 24 different primers were used to create 24 fusions in which the cpYFP was inserted at 6 different positions (after FeoB residues 36-41) and +/− ‘SAG’ or ‘GT’ linkers N- and C-terminal to the insertion point. A complete list of primers used in these constructions includes:

Primers for Generating the FeoB-cpYFP Chimera

Primers for Position #1-2 series: Forward primer 2 (no SAG linker): gactgccggctacaacagcgacaacgtc (SEQ ID NO:34); Reverse primer 2 (no GT linker): ttccttcctctcgacggtcacgttgtactccagcttgtgc (SEQ ID NO:35); Forward primer 2a (with SAG linker): gactgccggctctgctggttacaacagcgacaacgtc (SEQ ID NO:36); Reverse primer 2a (with GT linker): ctccttcctctcgacggtcactgttccgttgtactccagcttgtg (SEQ ID NO:37)

Primers for Position #2-6 Series: Forward primer 3 (no SAG linker): gactgccggcgtttacaacagcgacaacgtc (SEQ ID NO:38); Reverse primer 3 (no GT linker): ctccttcctctcgacggtgttgtactccagcttgtg (SEQ ID NO:39); Forward primer 3a (with SAG linker): gactgccggctctgctggtgtttacaacagcgacaacgtc (SEQ ID NO:40); Reverse primer 3a (with GT linker): ctccttcctctcgacggttgttccgttgtactccagcttgtg (SEQ ID NO:41)

Primers for Position #3-5 Series: Forward primer 4 (no SAG linker): gactgccggcgttacttacaacagcgacaacgtc (SEQ ID NO:42); Reverse primer 4 (no GT linker): ctccttcctctcgacgttgtactccagcttgtgc (SEQ ID NO:43); Forward primer 4a (with SAG linker): gactgccggcgttacttctgctggttacaacagcgacaacgtc (SEQ ID NO:44); Reverse primer 4a (with GT linker): ctccttcctctcgactgttccgttgtactccagcttgtgc (SEQ ID NO:45).

Primers for Position #4-1 series: Forward primer 1 (no SAG linker): gactgccggcgttactgtttacaacagcgacaacgtc (SEQ ID NO:46); Reverse primer 1 (no GT linker): ttctttacgttcgttgtactccagcttgtg (SEQ ID NO:47); Forward primer 1a (with SAG linker): gactgccggcgttactgtttctgctggttacaacagcgacaacgtc (SEQ ID NO:48); Reverse primer 1a (with GT linker): ttctttacgttcagtaccgttgtactccagcttgtg (SEQ ID NO:49).

Primers for Position #5-4 series: Forward primer 5 (no SAG linker): gactgccggcgttactgttgagtacaacagcgacaacgtc (SEQ ID NO:50); Reverse primer 5 (no GT linker): ctccttcctgttgtactccagcttgtgcc (SEQ ID NO:51); Forward primer 5a (with SAG linker): gactgccggcgttactgttgagtctgctggttacaacagcgacaacgtc (SEQ ID NO:52); Reverse primer 5a (with GT linker): ctccttccttgttccgttgtactccagcttgtgcc (SEQ ID NO:53).

Primers for Position #6-3 Series: Forward primer 6 (no SAG linker): gactgccggcgttactgttgagaggtacaacagcgacaacgtc (SEQ ID NO:54); Reverse primer (no GT linker): ttccttgttgtactccagcttgtgcccc (SEQ ID NO:55); Forward primer 6a (with SAG linker): gactgccggcgttactgttgagaggtctgctggttacaacagcgacaacgtc (SEQ ID NO:56); Reverse primer 6a (with GT linker): ttcctttgttccgttgtactccagcttgtgcccc (SEQ ID NO:57).

In certain aspects linkers can be encoded by tctgctggt (SEQ ID NO:58)—SAG and agtacc (SEQ ID NO:59), tgttcc (SEQ ID NO:60)—GT.

Primers used to move the FeoB-cpYFP insert from pRSET vector to the pET vector for expression: Forward Primer, NdeI site: cattgcatatgaaaaaactgacaatcggactgatcg (SEQ ID NO:61); Reverse Primer, XhoI: cggtactcgagttaggacacgacatcgc (SEQ ID NO:62).

To append mAmterine to the fusion, the mAmetrine DNA fragment and the pET vector containing the FeoB-cpYFP fusion were amplified with the primers below, cut with NheI and NdeI and then ligated together:

mAmetrine-(FeoB-cpYFP): For amplifying the pET plasmid with the FeoB-cpYFP insert: Forward primer, NheI: ggtggcgctagcggtggaaaaaaactgacaatcgg (SEQ ID NO:63); Reverse primer, NdeI site: gcgccccatatgaccttggaagtagaggttctc (SEQ ID NO:64).

For amplifying the mAmetrine fragment: Forward primer, NdeI: cattgcatatggtgagcaagggcgag (SEQ ID NO:65); Reverse primer, NheI: tataccgctagctccacccttgtacagctcgtc (SEQ ID NO:66)

(FeoB-cpYFP)-mAmetrine: For amplifying the pET plasmid mAmetrine insert: Forward primer, NheI: atgacagctagcggtggaatggtgagcaaggg (SEQ ID NO:67); Reverse primer, NdeI site: gcgccccatatgaccttggaagtagaggttctc (SEQ ID NO:68).

For amplifying the FeoB-cpYFP fragment: Forward primer, NdeI: cattgcatatgaaaaaactgacaatcggactgatcg (SEQ ID NO:69); Reverse primer, NheI: tataccgctagctccaccggacacgacatcgc (SEQ ID NO:70)

Primers used to ligate the mAmetrine to the N- or C-terminal of the FeoB-cpYFP chimera (mAmetrine-(FeoB-cpYFP))

For amplifying the pET plasmid with the FeoB-cpYFP insert: Forward primer, NheI: ggtggcgctagcggtggaaaaaaactgacaatcgg (SEQ ID NO:71); Reverse primer, NdeI site: gcgccccatatgaccttggaagtagaggttctc (SEQ ID NO:72).

For amplifying the mAmetrine fragment: Forward primer, NdeI: cattgcatatggtgagcaagggcgag (SEQ ID NO:73); Reverse primer, NheI: tataccgctagctccacccttgtacagctcgtc (SEQ ID NO:74).

(FeoB-cpYFP)-mAmetrine—For amplifying the pET plasmid mAmetrine insert: Forward primer, NheI: atgacagctagcggtggaatggtgagcaaggg (SEQ ID NO:75); Reverse primer, NdeI site: gcgccccatatgaccttggaagtagaggttctc (SEQ ID NO:76).

For amplifying the FeoB-cpYFP fragment: Forward primer, NdeI: cattgcatatgaaaaaactgacaatcggactgatcg (SEQ ID NO:77); Reverse primer, NheI: tataccgctagctccaccggacacgacatcgc (SEQ ID NO:78).

Site-Directed Mutagenesis of the FeoB GI Region.

P12G: Primer: caatcggactgatcggcaatggtaatagcggcaaaactacac (SEQ ID NO:79); Primer_rc: gtgtagttttgccgctattaccattgccgatcagtccgattg (SEQ ID NO:80)

P12G+S14A (use P12G as template): Primer: gatcggcaatggtaatgccggcaaaactacactg (SEQ ID NO:81); Primer_rc: cagtgtagttttgccggcattaccattgccgatc (SEQ ID NO:82)

P12G+T175 (use P12G as template): Primer: ggtaatagcggcaaatctacactgttcaaccagctg (SEQ ID NO:83); Primer_rc: cagctggttgaacagtgtagatttgccgctattacc (SEQ ID NO:84).

Triple (use P12G as template): Primer: gatcggcaatggtaatgccggcaaatctacactgttcaaccagctg (SEQ ID NO:85); Primer_rc: cagctggttgaacagtgtagatttgccggcattaccattgccgatc (SEQ ID NO:86).

Subcloning into the pGW1 Mammalian Expression Vector

mAmetrine-(FeoB-cpYFP): Forward, HindIII: gattagaagcttatggtgagcaagggcgaggag (SEQ ID NO:87); Reverse, EcoRI: ctgagcgaattcttaggacacgacatcgca (SEQ ID NO:88).

(FeoB-cpYFP)-mAmetrine: Forward, HindIII: gctcagaagcttatgaaaaaactgacaatcgg (SEQ ID NO:89); Reverse, EcoRI: ctgagcgaattcttacttgtacagctcgtcc (SEQ ID NO:90)

### Example 2

### In Vitro Validation

Expression and purification: The FA/AF11A protein construct in a modified pET32 vector was used to transform BL21 (DE3) cells. Protein expression was induced with IPTG at a final concentration of 1 mM when the cell OD₆₀₀ reached 0.6-0.8. Cells were grown for a further 12-14 hrs at 30° C. overnight. Cells were harvested in the morning, suspended in 50 mM TrisHCl, pH 8.3, 2 mM NaCl, 0.1% (v/v) Tween-20, 10 mM imidazole, 1 mM PMSF (lysis buffer), and lysed by sonication. Cell debris was removed by spinning at 17000 rpm for 30 minutes at 4° C. in a Sorvall RC-5C centrifuge equipped with a SS-34 rotor. The lysate, which is bright yellow, was applied to a NiNTA column equilibrated with the same lysis buffer. The loaded NiNTA column was washed with 5 volumes of the lysis buffer followed by 2-volumes of 50 mM TrisHCl, pH8.3, 400 mM NaCl, 50 mM imidazole. Then the protein was eluted in 2 volumes of 50 mM TrisHCl, pH 7.5, 100 mM NaCl, 300 mM imidazole. The protein was dialyzed overnight against the binding buffer: 25 mM TrisHCl, pH7.5, 100 mM KCl, 5 mM MgCl₂, 10% (v/v) glycerol. Protein concentration was determined with a BCA assay-kit from Invitrogen using bovine serum albumin for the calibration curve.

GTP Binding Analysis:

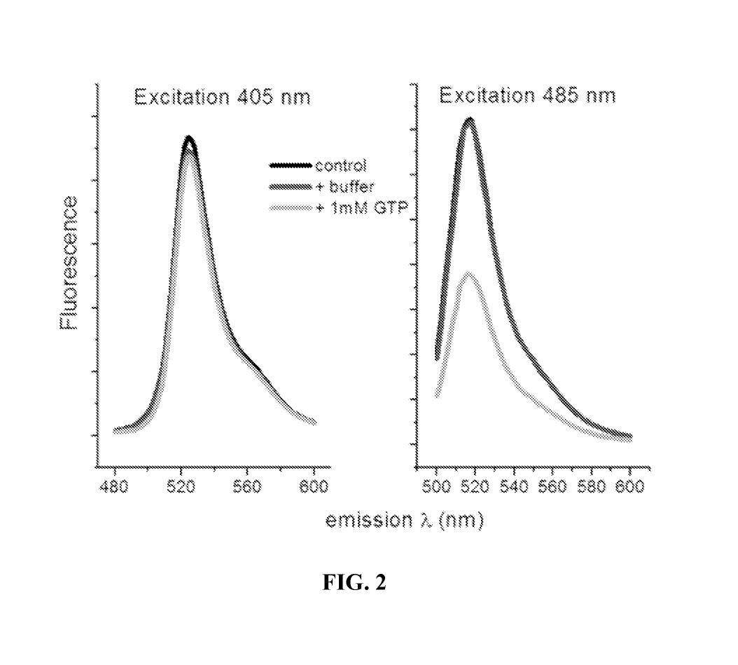

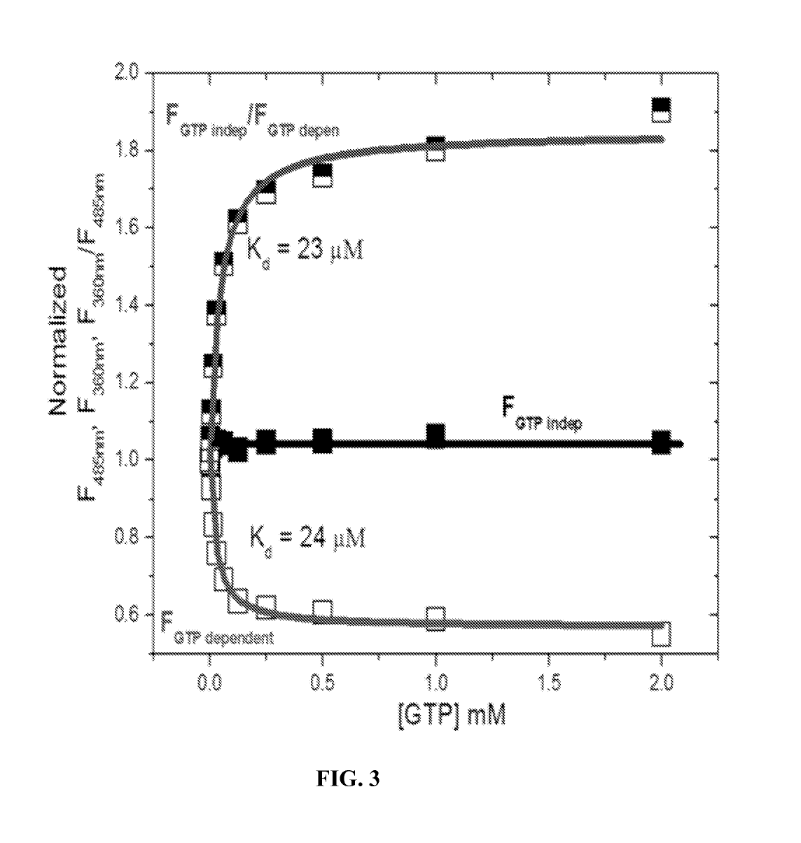

Fluorescent emission spectra of FA/AF11A were obtained at ˜1 μM in binding buffer and 3.0 ml in a quartz cuvette. Emission data at 485 nm excitation followed by one at 405 nm were collected sequentially. Emission and excitation bandwidths were set at 2 nm. 30 μl of either buffer or a 100 mM GTP stock solution was added and data collected again. The results are shown in FIG. 2. Titrations with GTP, GDP, and ATP were carried out in a BioTeK plate reader on 96-well fluorescence plates from Whatman. Protein concentrations were 1 μM and ligand concentrations varied from 0 to 2 mM. FA/AF11A was excited at 485 nm and 360 nm with the emission collected at 528 nm. A typical result and its analysis is shown in FIG. 3. As there is no agreement on the absolute GTP concentrations inside cells (or they may be different for different cells or even during different stages of the same cell's life cycle), a series of GTP sensors with differing GTP affinities were constructed. The series of GTP sensors were obtained by replacement of key residues involved in GTP binding (PNSGKTT). The key residue(s) replacement and the resulting changes in ligand affinities along with the GTP sensors label are given in Table I. Of particular importance is the FA/AF AAA construct. This is a sensor that actually does not work due to its GTP binding site being altered. However this is a control sensor for use in in vivo situations to allow for pH effects on the fluorescent proteins.

TABLE I
GTP sensors and their properties

| Label^{*} | Sequence | GTP K_{d} (μM) | GDP K_{d} (μM) |
|---|---|---|---|
| 11A | PNSGKTT (SEQ ID NO: 6) | 21.8 | 306 |
| 12 | GNSGKTT (SEQ ID NO: 7 | 85.0 | 382 |
| 1214 | GNAGKTT (SEQ ID NO: 8) | 177 | 525 |
| 1217 | GNSGKST (SEQ ID NO: 9) | 275 | ~650 |
| Triple | GNAGKST (SEQ ID NO: 10) | 517 | ~1000 |
| AAA | PNAGAAT (SEQ ID NO: 11) | ND^{§} | ND |

^{*}As both the AF 11A and FA 11A have the same properties, the labels have omitted these letters.
^{§}not determined
Consensus of X₁NX₃GX₅X₆T, wherein X1 is G or P, X3 is S or A, X5 is K or A, X6 is T or S.

### Example 3

### In Vivo Validation

Transfection and Expression:

AF/FA GTP sensors were subcloned into a pGW1 mammalian cell expression vector with a cytomegalovirus (CMV) promoter. HEK293 cells were transfected with the plasmid DNA using the GenJet (SignaGen) transfection reagent according to the manufacturer's instructions. Cells were allowed to grow for ˜24 hrs before being treated with mycophenolic acid (MPA), which inhibits GTP synthesis. Cells were imaged 20 hrs after drug administration. All cell cultures were carried out with DMEM containing 10% FBS and penicillin/streptomycin and grown at 37° C. in an atmosphere with 5% CO₂. Passage number of cells was less than 30 for all experiments.

Imaging: Cells were imaged in a LabTek 8-chamber 1.5 coverslip-bottom culture-cum-imaging wells. Nikon Eclipse inverted epifluorescence microscope with Sedat Quad 89000 (Chroma Tech) filter along with Sutter instruments automated filter wheel controls was used for all imaging purposes. To ensure adequate signal, 2×2 binning was used. Fluorescein isothiocyanate (FITC) configuration was used with an exposure of ˜400-700 msec for the GTP sensitive (FY-core) signal. A custom configuration with an excitation wavelength of 380 nm, emission as for FITC, and exposure of ˜100-200 msec was used for the normalizer mAmetrine 1.1. Differential interference contrast (DIC) images were also obtained to ensure that the cells observed were healthy and normal.

Images were obtained individually without using the Nikon NIS software ratiometric collection mode. All image quantitative analyses were carried out with the public domain ImageJ (http://rsbweb.nih.gov/ij/) in conjunction with MS Excel and Origin 7. Briefly, region of interest (ROI) tool in ImageJ was used to obtain average intensities of the GTP-sensitive and GTP-insensitive signals from multiple cells (50-70). Two columns of intensities were obtained and corrected for background (obtained from cell-free ROI). Then the ratios of the two intensities were calculated along with the relevant statistics.

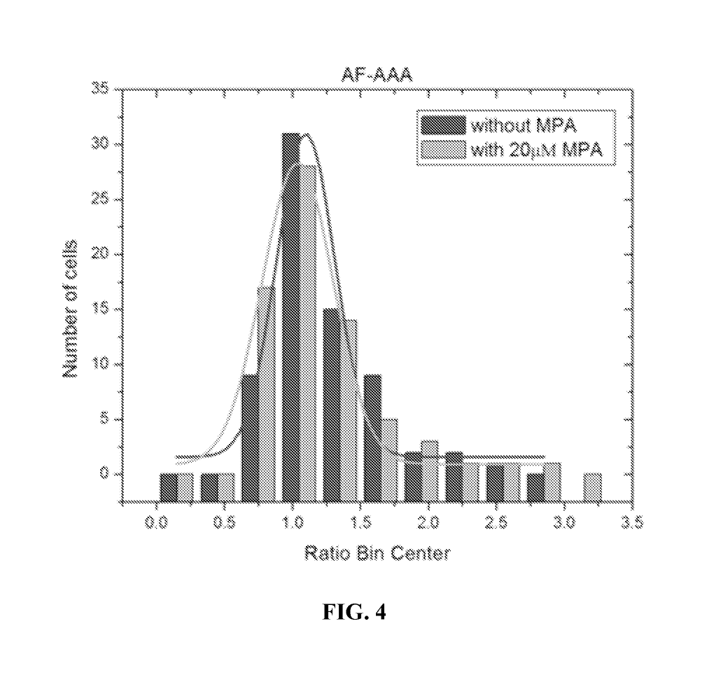

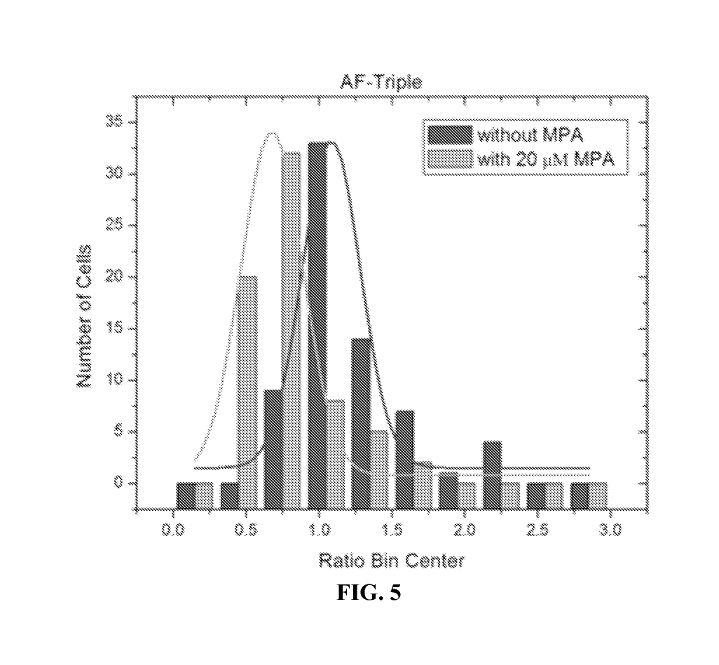

Pseudo-colored images overlaid on the DIC images of the cells in FIG. 4 show that there is a robust signal from both the GTP sensitive cpYFP (green) and the normalizer mAmetrine (red). Furthermore the two signals overlap perfectly as expected. Such an image is shown in FIG. 5—a simple merge of the two pseudo-colored images comparing the effect of MPA treatment. A study of the two panel shows that the cells treated with 20 μM MPA have a brighter green hue than those untreated. This is as expected; decrease in intracellular GTP upon MPA treatment leads to an increase in the signal from the cpYFP (green) which is inversely related to the GTP concentration (refer to FIGS. 2 and 3).

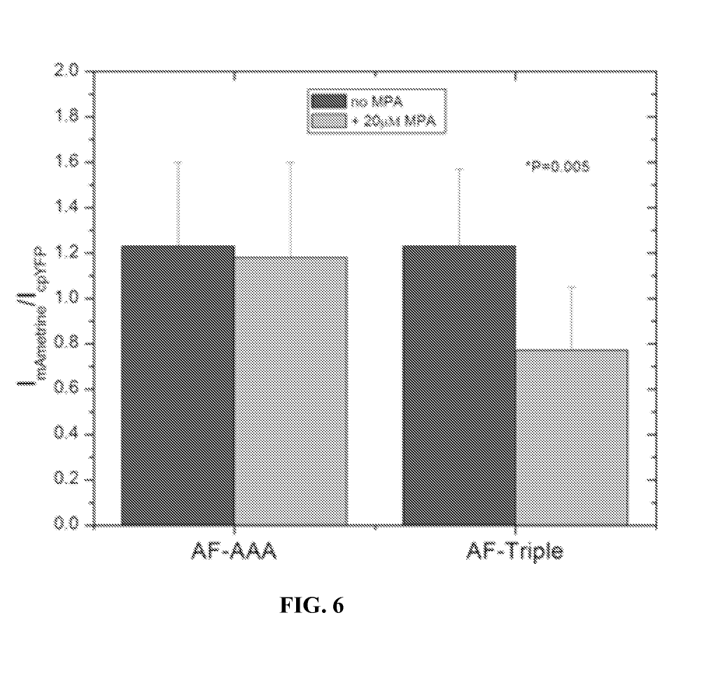

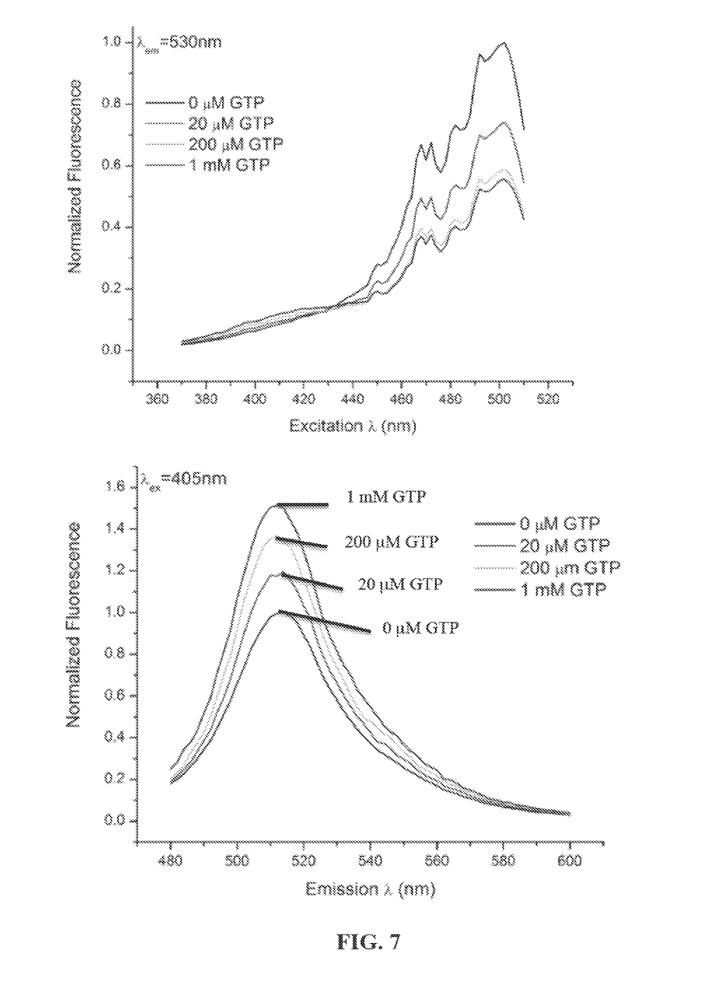

Results from quantitative analyses are shown in FIG. 6. As expected, for the GTP sensor construct AF-AAA, whose GTP sensing ability has been compromised, MPA treatment shows no effect. However for the AF-Triple construct, there is a distinct difference in the distribution of the ratio as well as the average value. The difference is statistically significant even though the error bars are rather large.

### Example 4

### 5A-5A Sensor

DNA encoding a cpYFP was inserted into the DNA encoding residues 35-40 of the FeoB G-protein domain (residues 1-260). Six insertion sites—spaced one codon apart—and 4 combinations of linker lengths between the N- and C-termini of the cpYFP and FeoB genes were used to construct 24 different fusion proteins. Nomenclature followed the form “FY1a-1a”, were the number designates the cpYFP insertion site, with “1” corresponding to insertion after FeoB residue 35 and “6” to insertion after residue 40, and where an “a” after the number indicates the presence of a ser-ala-gly or gly-thr linker at, respectively, the N- or C-terminus of the cpYFP.

The first generation GTP sensor uses 2 fluorescent proteins (FP): one whose fluorescence changed in response to GTP binding and one that did not. The fluorescence from the GTP insensitive protein provided a way to normalize the GTP-sensitive fluorescent signal. The second generation GTP sensor (e.g., 5a-5a sensor) uses one FP and takes advantage of the fact that GTP binding causes one part of its excitation spectrum to decrease, while another part decreases. The ratio of the fluorescence at these two excitation wavelengths is therefore internally normalized for changes in sensor expression levels. In addition, using a single FP provides certain advantages over a 2-FP sensor: (1.) A single FP sensor still generates a ratiometric signal because the binding of GTP causes a decrease in fluorescence when excited at longer wavelengths (˜500 nm) but a decrease in when excited at shorter (˜400 nm) wavelengths. This mitigates the effects of pH because one FP has one pKa value whereas two FPs may have two different pKa values. (2.) Having a single FP eliminates potential problems due to differences in the rates of maturation or proteolysis of the two FPs in the cells. (3.) The change in fluorescence ratio varies from 2-6 fold, depending on pH. This is substantially larger than the ratio change seen with the 2 FP sensor, which was always ≦2-fold.

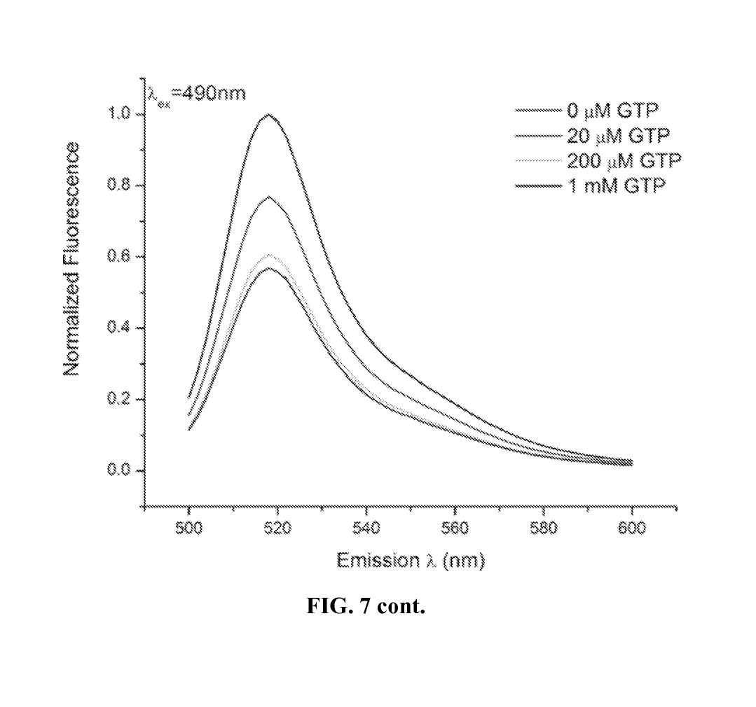

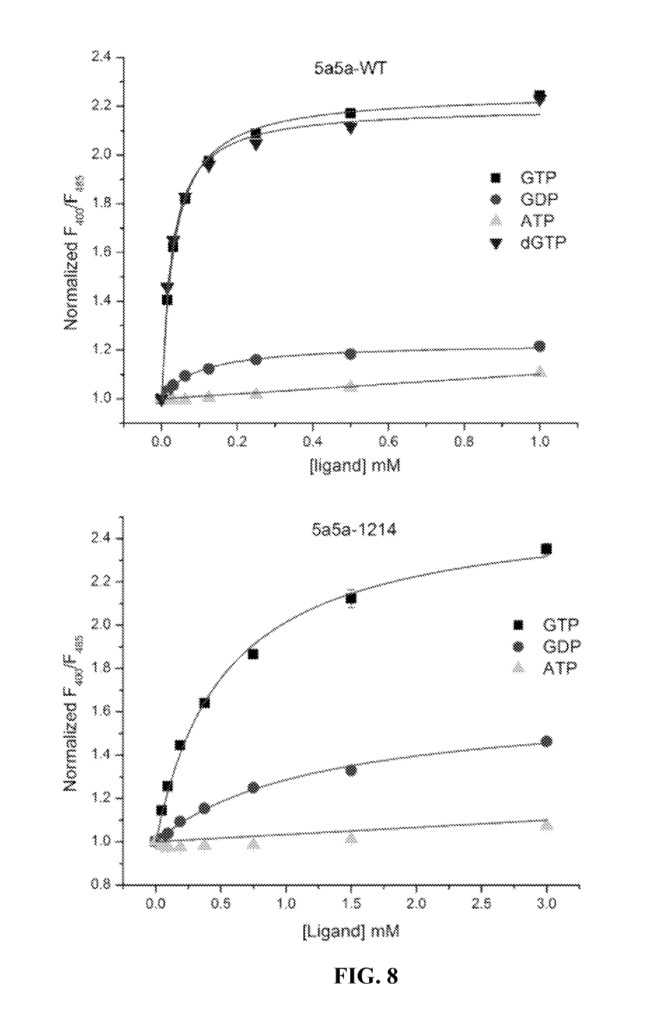

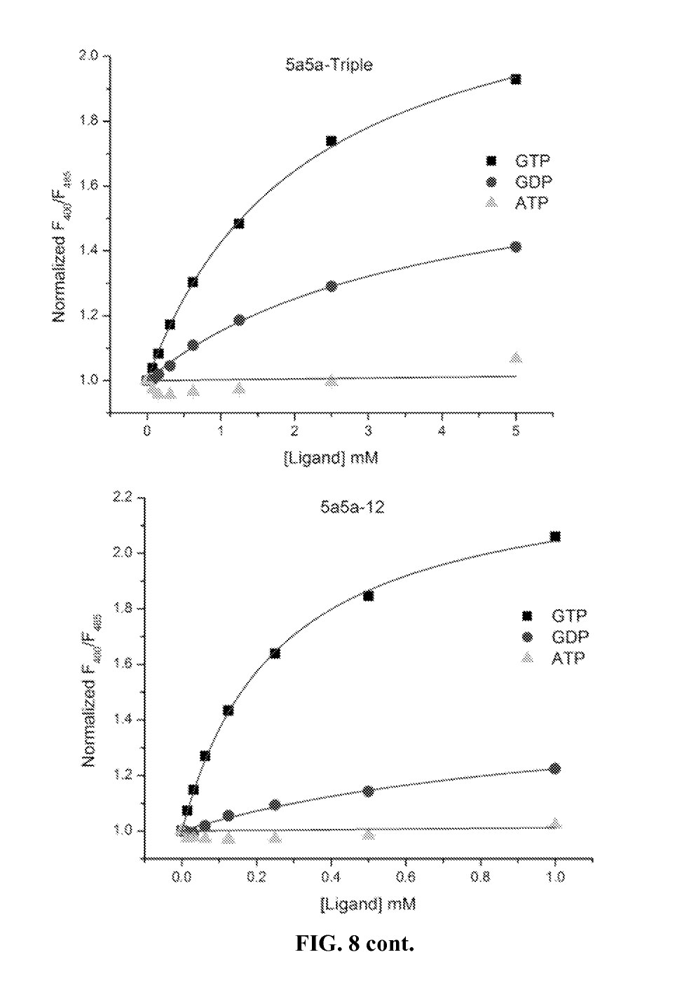

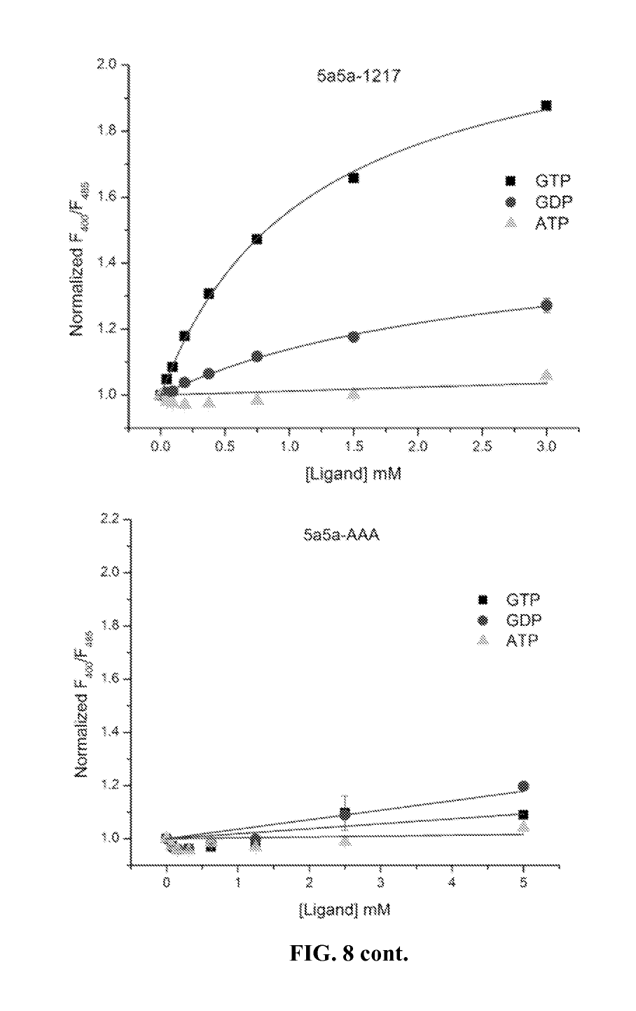

5a5a-GTP sensor (SEQ ID NO:14) and its in vitro characterization. The 5a5a sensor is responsive to GTP. The 5a5a sensor binds dGTP as well as, if not slightly better than GTP itself (FIG. 7 and FIG. 8). The curves look very similar but that is because of the different x-axis—with GTP up to 5 mM for low affinity sensors.

TABLE 2
Summary of 5a5a sensor data.

| Sample | GTP K_{eff} | GDP K_{eff} |
|---|---|---|
| 5a5a-WT | 33.2 ± 0.3 | 100 ± 6 |
| 5a5a-12 | 260 ± 20 | 1000 ± 200 |
| 5a5a-1214 | 528 ± 20 | 1280 ± 200 |
| 5a5a-1217 | 1150 ± 100 | 2770 ± 300 |
| 5a5a-Triple | 2300 ± 100 | 3700 ± 300 |
| 5a5a-AAA | N/A | N/A |

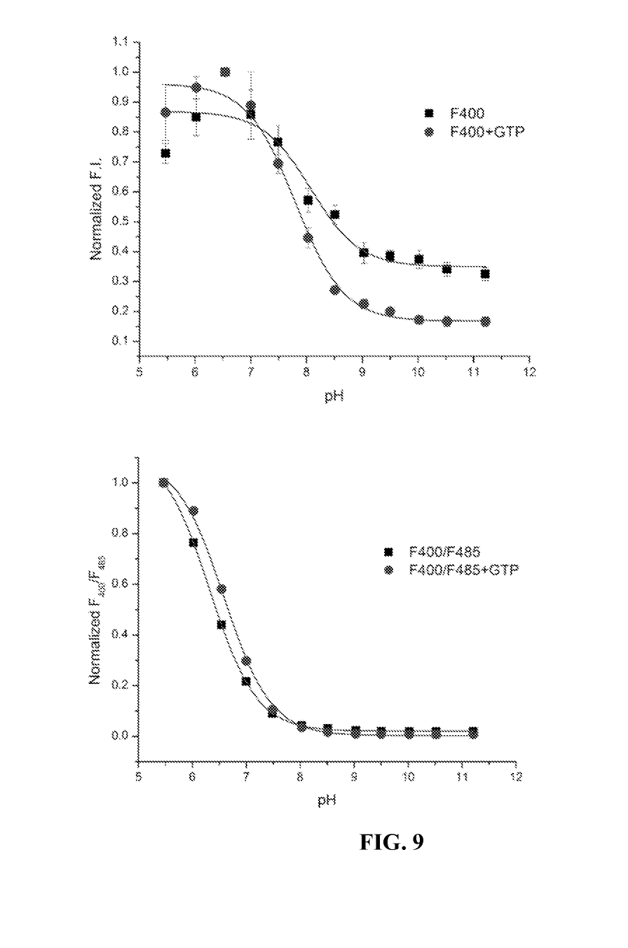

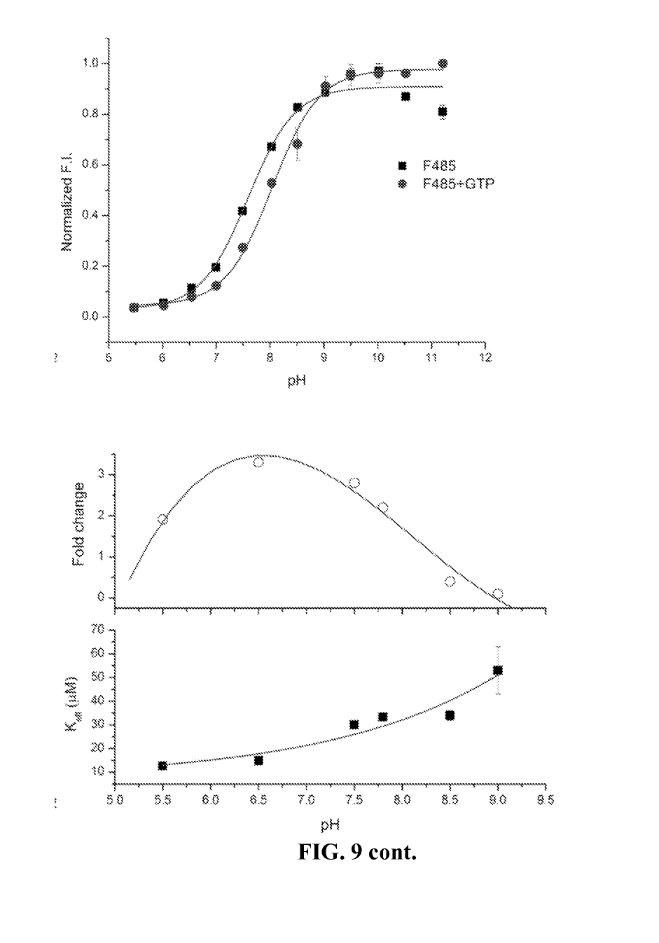

The fluorescence from the 400 and 485 nm excitations respond differently to pH and also GTP (FIG. 9). In the case of the 400 nm excitation, the pKa in the absence of GTP is 8.1 and decreases to 7.8 upon GTP addition. For the 485 nm excitation, these numbers are 7.6 to 8.0. For the 400/485 ratio, the pKa in the absence and presence of GTP are 6.3 and 6.6 respectively. The good thing about the sensor in the pH department is that the ratio is already flattening out around pH 7.5—so the pH is unlikely have a large effect unlike in our previous GEval constructs. Another interesting fact is how dependent the fold-change is on pH with a peak around pH 6.5. I have not thoroughly analyzed the individual 400 nm or 485 nm excitation changes but they are seem to be a bit complex and no pattern jumps out immediately. Anyway, the ratio of 400/485 is the important parameter to look at.

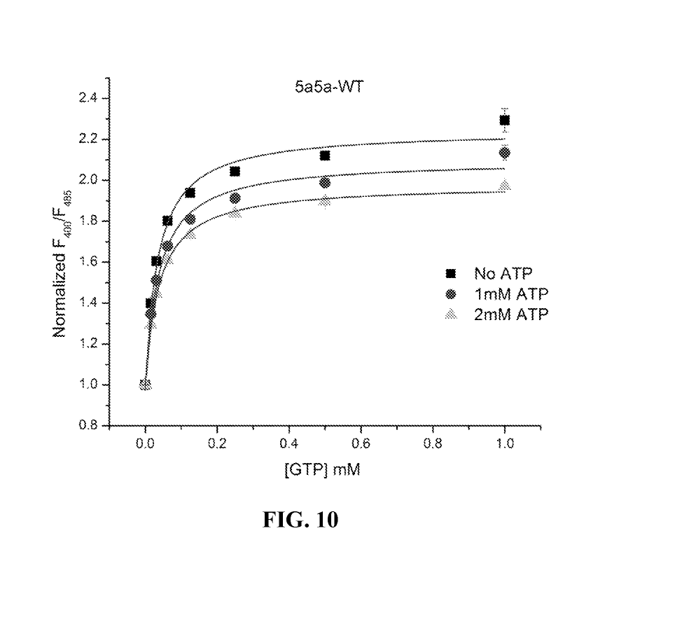

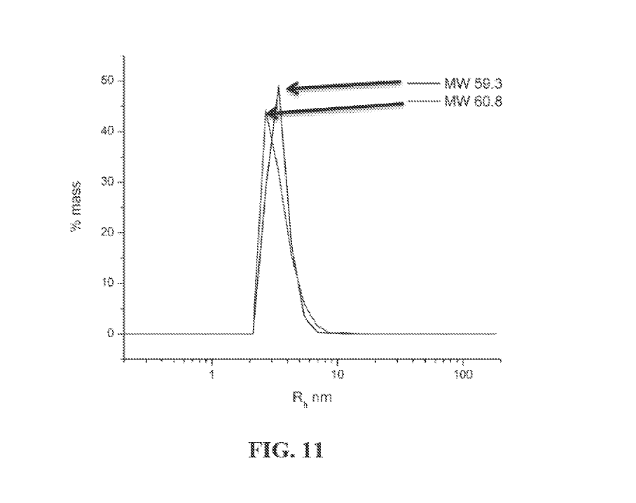

The K_{eff} increases from 35±1 μM to 38±2 μM for both 1 and 2 mM ATP. There is however a slight decrease in fold-change with increasing ATP concentration. (FIG. 10)

The 5a5a sensor exists as a monomer in solution. These are two independent samples at 50 μM each. The MW comes out to 59.3 and 60.8 kDa—very close to the calculated MW of ˜58.1 kDa (FIG. 11). The solution condition was taken as that of PBS (determines the viscosity for Diffusion Coeff calculation and hence the Rₕ) and the model as isotropic sphere (determines the MW calculation from the Diffusion Coeff via Rₕ). The % mass for both cases is 99.7 with 0.3 something else that has Rₕ around 10. The buffer for the experiment was 50 mM TrisHCl, pH7.8, 100 mM KCl, 5 mM MgCl2.

Consistent with the above DLS data is the SDS-PAGE gel confirms the MW and production of a monomeric 5a5a sensor. After Ni-NTA column the dominant band seen on SDAS-PAGE gel is a 5a5a sensor monomer—no need for further purification. The band runs as expected for a ˜60 kDa protein.

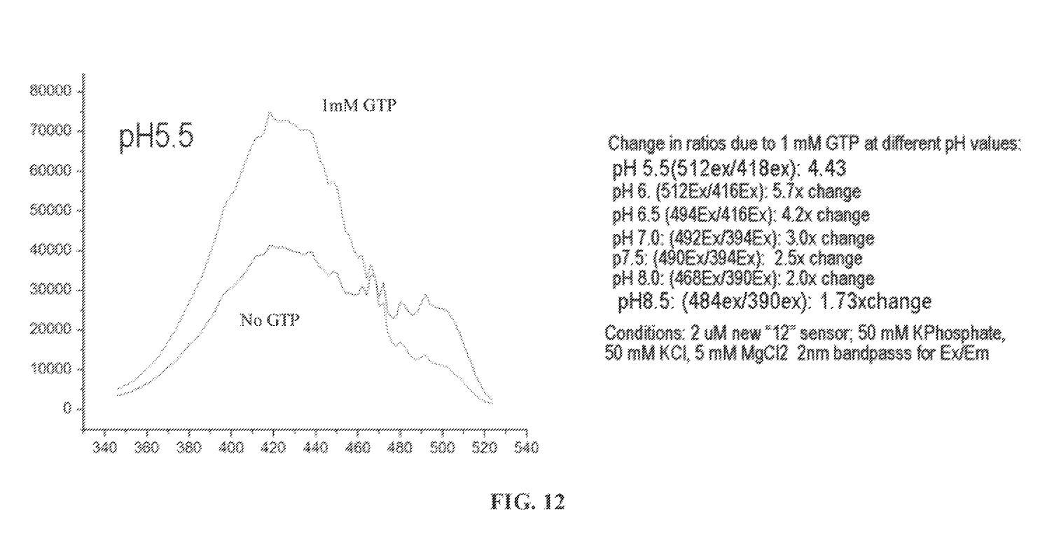

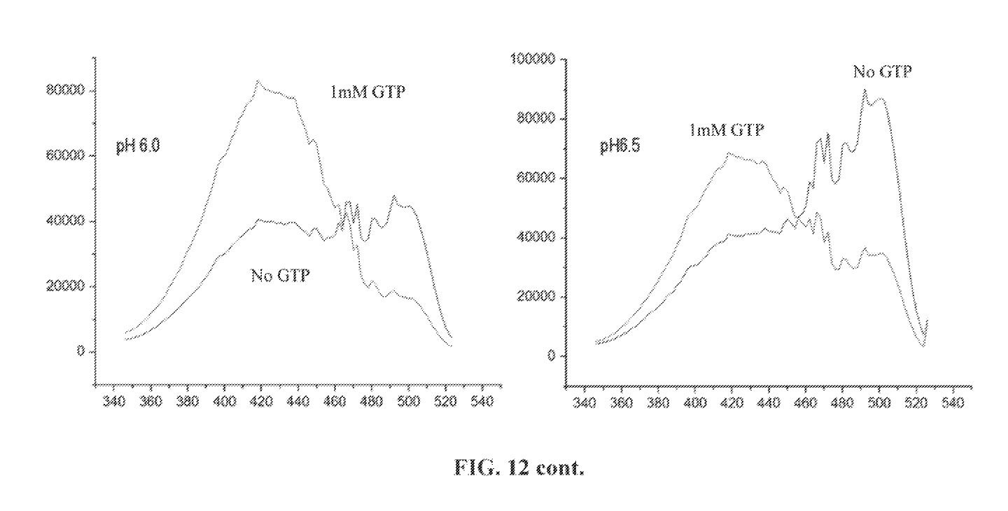

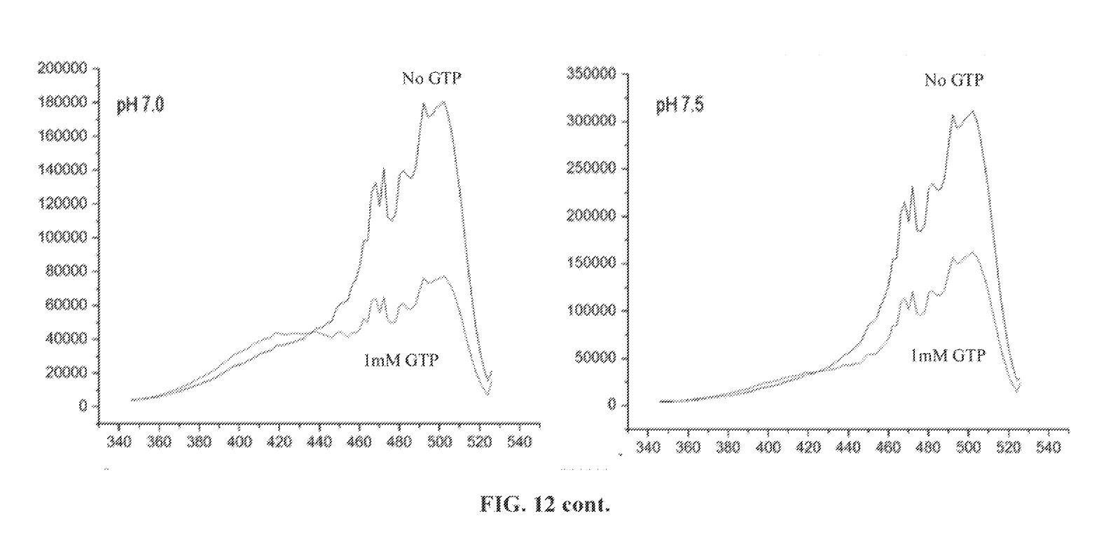

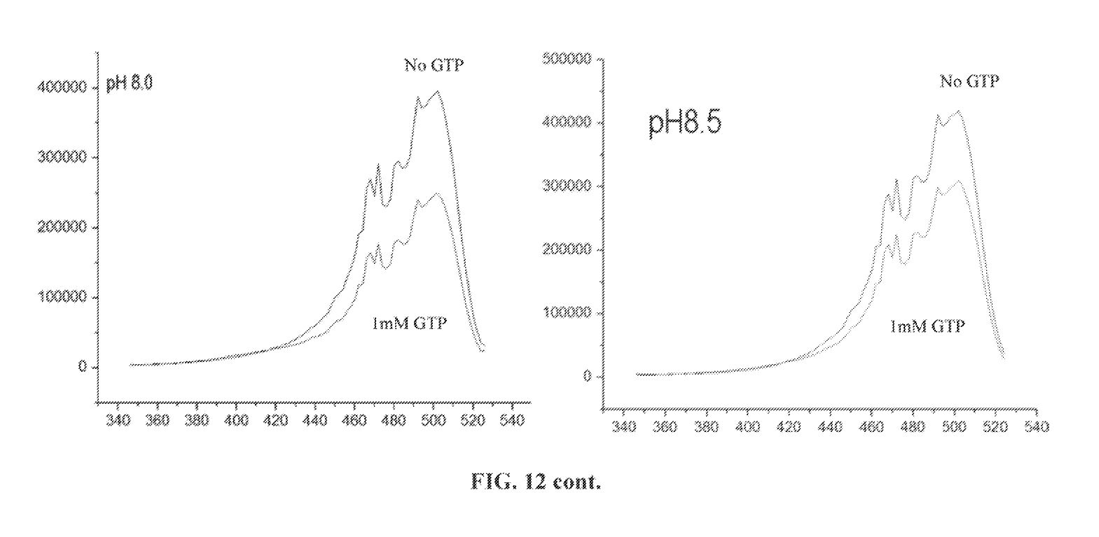

pH Effects and Extent of GTP-Driven Changes in Ratiometric Fluorescent Signal: What these scans reveal is that, relative to the previous 2-FP (mAmetrine+cpYFP) sensor, the 5a-5a sensor is superior in that: 1. It uses a single FP protein but still generates a ratiometric signal because the binding of GTP causes a decrease in fluorescence when excited at longer wavelengths (˜500 nm) but a decrease in when excited at shorter (˜400 nm) wavelengths. This mitigates the effects of pH (because we don't have to worry about the problem of having two FPs with different pKa values. It also eliminates potential problems due to differences in the rates of maturation or proteolysis of the two FPs in the cells. (FIG. 12)

1. A polypeptide comprising an amino terminal GTP binding domain coupled to a central conformation sensitive fluorescent protein domain that is coupled to a carboxy terminal guanine-dissociation inhibitor domain, wherein fluorescence of the polypeptide changes upon binding to GTP.

2. The polypeptide of claim 1, further comprising a terminal normalizer domain.

3. The polypeptide of claim 2, wherein the normalizer domain is positioned at the amino terminus.

4. The polypeptide of claim 1, wherein the polypeptide has an amino acid sequence that is 90% identical to SEQ ID NO:2 or SEQ ID NO:14.

5. The polypeptide of claim 1, wherein the polypeptide has an amino acid sequence of SEQ ID NO:2 or SEQ ID NO:14.

6. A cell expressing the polypeptide of claim 1.

7. An organism comprising the cell of claim 6.

8. A nucleic acid encoding the polypeptide of claim 1.

9. An expression vector comprising the nucleic acid of claim 9.

10. A cell comprising the nucleic acid of claim 8.

11. A cell comprising a nucleic acid of claim 8 integrated into the genome.

12. A kit comprising a polypeptide of claim 1 or a nucleic acid encoding the polypeptide of claim 1.

13. A method for measuring the GTP concentration in a cell comprising:
(a) providing a polypeptide sensor of claim 1; and
(b) measuring fluorescence from the sensor.


SEQUENCE LISTING (92 sequences)

SEQ ID NO: 1 - protein, 753 aa, Artificial Sequence
  Recombinant polypeptide
MVSKGEELFTGVVPILVELDGDVNGHRFSVRGEGEGDATNGKLTLKFICTSGELPVPWPTLVTTLSYGVQCFARYPDHMKQHDFFKSAMPEGYVQERTISFKDDGSYRTRAEVKFEGDTLVNRIELKGIDFKEDGNILGHKLEYNMNAWDAYITADKQKNGIKANFKIEHNVKDGGVQLADAYQQNTPIGDGSVLLPDNHYLSFQSKLFKDPNEQRDHMVLLEFVTAAGITPGMDELYKGGAGSGSGGKKLTIGLIGNPNSGKTTLFNQLTGSRQRVGNWAGVTVYNSDNVYITADKQKNGIKANFKIRHNIEDGGVQLADHYQQNTPIGDGPVLLPDNHYLSFQSKLSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNGTERKEGQFSTTDHQVTLVDLPGTYSLTTISSQTSLDEQIACHYILSGDADLLINVVDASNLERNLYLTLQLLELGIPCIVALNMLDIAEKQNIRIEIDALSARLGCPVIPLVSTRGRGIEALKLAIDRYKANENVELVHYAQPLLNEADSLAKVMPSDIPLKQRRWLGLQMLEGDIYSRAYAGEASQHLDAALARLRNEMDDPALHIADARYQCIAAICDVVS

SEQ ID NO: 2 - protein, 514 aa, Artificial Sequence
  Recombinant polypeptide
GGAGSGSGGKKLTIGLIGNPNSGKTTLFNQLTGSRQRVGNWAGVTVYNSDNVYITADKQKNGIKANFKIRHNIEDGGVQLADHYQQNTPIGDGPVLLPDNHYLSFQSKLSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNGTERKEGQFSTTDHQVTLVDLPGTYSLTTISSQTSLDEQIACHYILSGDADLLINVVDASNLERNLYLTLQLLELGIPCIVALNMLDIAEKQNIRIEIDALSARLGCPVIPLVSTRGRGIEALKLAIDRYKANENVELVHYAQPLLNEADSLAKVMPSDIPLKQRRWLGLQMLEGDIYSRAYAGEASQHLDAALARLRNEMDDPALHIADARYQCIAAICDVVS

SEQ ID NO: 3 - protein, 260 aa, Artificial Sequence
  Recombinant polypeptide
MKKLTIGLIGNPNSGKTTLFNQLTGSRQRVGNWAGVTVERKEGQFSTTDHQVTLVDLPGTYSLTTISSQTSLDEQIACHYILSGDADLLINVVDASNLERNLYLTLQLLELGIPCIVALNMLDIAEKQNIRIEIDALSARLGCPVIPLVSTRGRGIEALKLAIDRYKANENVELVHYAQPLLNEADSLAKVMPSDIPLKQRRWLGLQMLEGDIYSRAYAGEASQHLDAALARLRNEMDDPALHIADARYQCIAAICDVVS

SEQ ID NO: 4 - protein, 244 aa, Artificial Sequence
  Recombinant polypeptide
YNSDNVYITADKQKNGIKANFKIRHNIEDGGVQLADHYQQNTPIGDGPVLLPDNHYLSFQSKLSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYN

SEQ ID NO: 5 - protein, 224 aa, Artificial Sequence
  Recombinant polypeptide
GTERKEGQFSTTDHQVTLVDLPGTYSLTTISSQTSLDEQIACHYILSGDADLLINVVDASNLERNLYLTLQLLELGIPCIVALNMLDIAEKQNIRIEIDALSARLGCPVIPLVSTRGRGIEALKLAIDRYKANENVELVHYAQPLLNEADSLAKVMPSDIPLKQRRWLGLQMLEGDIYSRAYAGEASQHLDAALARLRNEMDDPALHIADARYQCIAAICDVVS

SEQ ID NO: 6 - protein, 7 aa, Artificial Sequence
  synthetic peptide
PNSGKTT

SEQ ID NO: 7 - protein, 7 aa, Artificial Sequence
  Synthetic peptide
GNSGKTT

SEQ ID NO: 8 - protein, 7 aa, Artificial Sequence
  Synthetic peptide
GNAGKTT

SEQ ID NO: 9 - protein, 7 aa, Artificial Sequence
  Synthetic peptide
GNSGKST

SEQ ID NO: 10 - protein, 7 aa, Artificial Sequence
  Synthetic peptide
GNAGKST

SEQ ID NO: 11 - protein, 7 aa, Artificial Sequence
  Synthetic peptide
PNAGAAT

SEQ ID NO: 12 - protein, 7 aa, Artificial Sequence
  Synthetic peptide
XNXGKXT

SEQ ID NO: 13 - protein, 239 aa, Artificial Sequence
  Recombinant polypeptide
MVSKGEELFTGVVPILVELDGDVNGHRFSVRGEGEGDATNGKLTLKFICTSGELPVPWPTLVTTLSYGVQCFARYPDHMKQHDFFKSAMPEGYVQERTISFKDDGSYRTRAEVKFEGDTLVNRIELKGIDFKEDGNILGHKLEYNMNAWDAYITADKQKNGIKANFKIEHNVKDGGVQLADAYQQNTPIGDGSVLLPDNHYLSFQSKLFKDPNEQRDHMVLLEFVTAAGITPGMDELYK

SEQ ID NO: 14 - protein, 509 aa, Artificial Sequence
  Recombinant polypeptide
MKKLTIGLIGNPNSGKTTLFNQLTGSRQRVGNWAGVTVSAGYNSDNVYITADKQKNGIKANFKIRHNIEDGGVQLADHYQQNTPIGDGPVLLPDNHYLSFQSKLSKDPNEKRDHMVLLEFVTAAGITLGMDELYKGGSGGMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNGTERKPGQFSTTDHQVTLVDLPGTYSLTTISSQTSLDEQIACHYILSGDADLLINVVDASNLERNLYLTLQLLELGIPCIVALNMLDIAEKQNIRIEIDALSARLGCPVIPLVSTRGRGIEALKLAIDRYKANENVELVHYAQPLLNEADSLAKVMPSDIPLKQRRWLGLQMLEGDIYSRAYAGEASQHLDAALARLRNEMDDPALHIADARYQCIAAICDVVS

SEQ ID NO: 15 - DNA, 792 bp, Artificial Sequence
  Recombinant nucleic acid
catatgaaaaaactgacaatcggactgatcggcaatcctaatagcggcaaaactacactgttcaaccagctgacagggtctcggcagcgggtcggaaactgggccggcgtgaccgtcgagaggaagcccgggcagttctctaccacagaccaccaggtgacactggtcgatctgccagggacttactccctgactaccattagctcccagacatctctggacgaacagattgcctgccattatatcctgagcggagacgctgatctgctgatcaatgtggtcgatgcatccaacctggagcggaatctgtacctgactctgcagctgctggaactgggcattccctgcatcgtggctctgaacatgctggacattgcagagaaacagaatatcagaattgaaatcgatgctctgagtgcaaggctggggtgtcccgtgatccctctggtctcaacaaggggccgaggaattgaggccctgaagctggctatcgaccgctacaaagcaaacgagaatgtggaactggtccactatgcccagcccctgctgaacgaagcagactctctggccaaagtgatgccaagtgatattcccctgaaacagcggagatggctggggctgcagatgctggagggagacatctacagccgggcctacgccggggaagcaagccagcatctggatgccgctctggctcgactgcggaatgagatggacgatcctgccctgcatattgctgacgcccgctaccagtgtattgctgctatttgcgatgtcgtgtcctaactcgag

SEQ ID NO: 16 - DNA, 732 bp, Artificial Sequence
  Recombinant nucleic acid
tacaacagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcttccagtccaagctgagcaaagaccccaacgagaagcgcgatcacatggtcctgctggagttcgtgaccgccgccgggatcactctcggcatggacgagctgtacaagggcggttccggaggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 17 - DNA, 37 bp, Artificial Sequence
  Synthetic oligonucleotide
gactgccggcgttactgtttacaacagcgacaacgtc

SEQ ID NO: 18 - protein, 12 aa, Artificial Sequence
  Synthetic peptide
TAGVTVYNSDNV

SEQ ID NO: 19 - DNA, 46 bp, Artificial Sequence
  Synthetic oligonucleotide
gactgccggcgttactgtttctgctggttacaacagcgacaacgtc

SEQ ID NO: 20 - protein, 15 aa, Artificial Sequence
  Synthetic peptide
TAGVTVSAGYNSDNV

SEQ ID NO: 21 - DNA, 30 bp, Artificial Sequence
  Synthetic oligonucleotide
ttctttacgttcgttgtactccagcttgtg

SEQ ID NO: 22 - protein, 10 aa, Artificial Sequence
  synthetic peptide
HKLEYNERKE

SEQ ID NO: 23 - DNA, 36 bp, Artificial Sequence
  Synthetic oligonucleotide
ttctttacgttcagtaccgttgtactccagcttgtg

SEQ ID NO: 24 - protein, 12 aa, Artificial Sequence
  Synthetic peptide
HKLEYNGTERKE

SEQ ID NO: 25 - DNA, 31 bp, Artificial Sequence
  Synthetic oligonucleotide
gactgccggcgtttacaacagcgacaacgtc

SEQ ID NO: 26 - protein, 10 aa, Artificial Sequence
  Synthetic peptide
TAGVYNSDNV

SEQ ID NO: 27 - DNA, 40 bp, Artificial Sequence
  Synthetic oligonucleotide
gactgccggctctgctggtgtttacaacagcgacaacgtc

SEQ ID NO: 28 - protein, 13 aa, Artificial Sequence
  Synthetic peptide
TAGVSAGYNSDNV

SEQ ID NO: 29 - DNA, 36 bp, Artificial Sequence
  Synthetic oligonucleotide
ctccttcctctcgacggtgttgtactccagcttgtg

SEQ ID NO: 30 - protein, 12 aa, Artificial Sequence
  Synthetic peptide
HKLEYNTVERKE

SEQ ID NO: 31 - DNA, 42 bp, Artificial Sequence
  Synthetic oligonucleotide
ctccttcctctcgacggttgttccgttgtactccagcttgtg

SEQ ID NO: 32 - protein, 14 aa, Artificial Sequence
  Synthetic peptide
HKLEYNGTTVERKE

SEQ ID NO: 33 - protein, 6 aa, Artificial Sequence
  Synthetic peptide
VTVERK

SEQ ID NO: 34 - DNA, 28 bp, Artificial Sequence
  Synthetic oligonucleotide
gactgccggctacaacagcgacaacgtc

SEQ ID NO: 35 - DNA, 40 bp, Artificial Sequence
  Synthetic oligonucleotide
ttccttcctctcgacggtcacgttgtactccagcttgtgc

SEQ ID NO: 36 - DNA, 37 bp, Artificial Sequence
  Synthetic oligonucleotide
gactgccggctctgctggttacaacagcgacaacgtc

SEQ ID NO: 37 - DNA, 45 bp, Artificial Sequence
  Synthetic oligonucleotide
ctccttcctctcgacggtcactgttccgttgtactccagcttgtg

SEQ ID NO: 38 - DNA, 31 bp, Artificial Sequence; identical to SEQ ID NO: 25
  Synthetic oligonucleotide

SEQ ID NO: 39 - DNA, 36 bp, Artificial Sequence; identical to SEQ ID NO: 29
  Synthetic oligonucleotide

SEQ ID NO: 40 - DNA, 40 bp, Artificial Sequence; identical to SEQ ID NO: 27
  Synthetic oligonucleotide

SEQ ID NO: 41 - DNA, 42 bp, Artificial Sequence; identical to SEQ ID NO: 31
  Synthetic oligonucleotide

SEQ ID NO: 42 - DNA, 34 bp, Artificial Sequence
  Synthetic oligonucleotide
gactgccggcgttacttacaacagcgacaacgtc

SEQ ID NO: 43 - DNA, 34 bp, Artificial Sequence
  Synthetic oligonucleotide
ctccttcctctcgacgttgtactccagcttgtgc

SEQ ID NO: 44 - DNA, 43 bp, Artificial Sequence
  Synthetic oligonucleotide
gactgccggcgttacttctgctggttacaacagcgacaacgtc

SEQ ID NO: 45 - DNA, 40 bp, Artificial Sequence
  Synthetic oligonucleotide
ctccttcctctcgactgttccgttgtactccagcttgtgc

SEQ ID NO: 46 - DNA, 37 bp, Artificial Sequence; identical to SEQ ID NO: 17
  Synthetic oligonucleotide

SEQ ID NO: 47 - DNA, 30 bp, Artificial Sequence; identical to SEQ ID NO: 21
  Synthetic oligonucleotide

SEQ ID NO: 48 - DNA, 46 bp, Artificial Sequence; identical to SEQ ID NO: 19
  Synthetic oligonucleotide

SEQ ID NO: 49 - DNA, 36 bp, Artificial Sequence; identical to SEQ ID NO: 23
  Synthetic oligonucleotide

SEQ ID NO: 50 - DNA, 40 bp, Artificial Sequence
  Synthetic oligonucleotide
gactgccggcgttactgttgagtacaacagcgacaacgtc

SEQ ID NO: 51 - DNA, 29 bp, Artificial Sequence
  Synthetic oligonucleotide
ctccttcctgttgtactccagcttgtgcc

SEQ ID NO: 52 - DNA, 49 bp, Artificial Sequence
  Synthetic oligonucleotide
gactgccggcgttactgttgagtctgctggttacaacagcgacaacgtc

SEQ ID NO: 53 - DNA, 35 bp, Artificial Sequence
  Synthetic oligonucleotide
ctccttccttgttccgttgtactccagcttgtgcc

SEQ ID NO: 54 - DNA, 43 bp, Artificial Sequence
  Synthetic oligonucleotide
gactgccggcgttactgttgagaggtacaacagcgacaacgtc

SEQ ID NO: 55 - DNA, 28 bp, Artificial Sequence
  Synthetic oligonucleotide
ttccttgttgtactccagcttgtgcccc

SEQ ID NO: 56 - DNA, 52 bp, Artificial Sequence
  Synthetic oligonucleotide
gactgccggcgttactgttgagaggtctgctggttacaacagcgacaacgtc

SEQ ID NO: 57 - DNA, 34 bp, Artificial Sequence
  Synthetic oligonucleotide
ttcctttgttccgttgtactccagcttgtgcccc

SEQ ID NO: 58 - DNA, 9 bp, Artificial Sequence
  Synthetic oligonucleotide
tctgctggt

SEQ ID NO: 59 - DNA, 6 bp, Artificial Sequence
  Synthetic oligonucleotide
agtacc

SEQ ID NO: 60 - DNA, 6 bp, Artificial Sequence
  Synthetic oligonucleotide
tgttcc

SEQ ID NO: 61 - DNA, 36 bp, Artificial Sequence
  Synthetic oligonucleotide
cattgcatatgaaaaaactgacaatcggactgatcg

SEQ ID NO: 62 - DNA, 28 bp, Artificial Sequence
  Synthetic oligonucleotide
cggtactcgagttaggacacgacatcgc

SEQ ID NO: 63 - DNA, 35 bp, Artificial Sequence
  Synthetic oligonucleotide
ggtggcgctagcggtggaaaaaaactgacaatcgg

SEQ ID NO: 64 - DNA, 33 bp, Artificial Sequence
  Synthetic oligonucleotide
gcgccccatatgaccttggaagtagaggttctc

SEQ ID NO: 65 - DNA, 26 bp, Artificial Sequence
  Synthetic oligonucleotide
cattgcatatggtgagcaagggcgag

SEQ ID NO: 66 - DNA, 33 bp, Artificial Sequence
  Synthetic oligonucleotide
tataccgctagctccacccttgtacagctcgtc

SEQ ID NO: 67 - DNA, 32 bp, Artificial Sequence
  Synthetic oligonucleotide
atgacagctagcggtggaatggtgagcaaggg

SEQ ID NO: 68 - DNA, 33 bp, Artificial Sequence; identical to SEQ ID NO: 64
  Synthetic oligonucleotide

SEQ ID NO: 69 - DNA, 36 bp, Artificial Sequence; identical to SEQ ID NO: 61
  Synthetic oligonucleotide

SEQ ID NO: 70 - DNA, 32 bp, Artificial Sequence
  Synthetic oligonucleotide
tataccgctagctccaccggacacgacatcgc

SEQ ID NO: 71 - DNA, 35 bp, Artificial Sequence; identical to SEQ ID NO: 63
  Synthetic oligonucleotide

SEQ ID NO: 72 - DNA, 33 bp, Artificial Sequence; identical to SEQ ID NO: 64
  Synthetic oligonucleotide

SEQ ID NO: 73 - DNA, 26 bp, Artificial Sequence
  Synthetic oligonucleotide
cattgcatatggtgagcaagggcgag

SEQ ID NO: 74 - DNA, 33 bp, Artificial Sequence; identical to SEQ ID NO: 66
  Synthetic oligonucleotide

SEQ ID NO: 75 - DNA, 32 bp, Artificial Sequence; identical to SEQ ID NO: 67
  Synthetic oligonucleotide

SEQ ID NO: 76 - DNA, 33 bp, Artificial Sequence; identical to SEQ ID NO: 64
  Synthetic oligonucleotide

SEQ ID NO: 77 - DNA, 36 bp, Artificial Sequence; identical to SEQ ID NO: 61
  Synthetic oligonucleotide

SEQ ID NO: 78 - DNA, 32 bp, Artificial Sequence; identical to SEQ ID NO: 70
  Synthetic oligonucleotide

SEQ ID NO: 79 - DNA, 42 bp, Artificial Sequence
  Synthetic oligonucleotide
caatcggactgatcggcaatggtaatagcggcaaaactacac

SEQ ID NO: 80 - DNA, 42 bp, Artificial Sequence
  Synthetic oligonucleotide
gtgtagttttgccgctattaccattgccgatcagtccgattg

SEQ ID NO: 81 - DNA, 34 bp, Artificial Sequence
  Synthetic oligonucleotide
gatcggcaatggtaatgccggcaaaactacactg

SEQ ID NO: 82 - DNA, 34 bp, Artificial Sequence
  Synthetic oligonucleotide
cagtgtagttttgccggcattaccattgccgatc

SEQ ID NO: 83 - DNA, 36 bp, Artificial Sequence
  Synthetic oligonucleotide
ggtaatagcggcaaatctacactgttcaaccagctg

SEQ ID NO: 84 - DNA, 36 bp, Artificial Sequence
  Synthetic oligonucleotide
cagctggttgaacagtgtagatttgccgctattacc

SEQ ID NO: 85 - DNA, 46 bp, Artificial Sequence
  Synthetic oligonucleotide
gatcggcaatggtaatgccggcaaatctacactgttcaaccagctg

SEQ ID NO: 86 - DNA, 46 bp, Artificial Sequence
  Synthetic oligonucleotide
cagctggttgaacagtgtagatttgccggcattaccattgccgatc

SEQ ID NO: 87 - DNA, 33 bp, Artificial Sequence
  Synthetic oligonucleotide
gattagaagcttatggtgagcaagggcgaggag

SEQ ID NO: 88 - DNA, 30 bp, Artificial Sequence
  Synthetic oligonucleotide
ctgagcgaattcttaggacacgacatcgca

SEQ ID NO: 89 - DNA, 32 bp, Artificial Sequence
  Synthetic oligonucleotide
gctcagaagcttatgaaaaaactgacaatcgg

SEQ ID NO: 90 - DNA, 31 bp, Artificial Sequence
  Synthetic oligonucleotide
ctgagcgaattcttacttgtacagctcgtcc

SEQ ID NO: 91 - protein, 5 aa, Artificial Sequence
  synthetic peptide
AGVTV

SEQ ID NO: 92 - protein, 4 aa, Artificial Sequence
  Synthetic peptide
ERKE